(19) United States
(12) Patent Application Publication
(10) Pub. No.: US 2012/0190091 A1
(43) Pub. Date: Jul. 26, 2012

(54) LIQUID-PHASE AND VAPOR-PHASE DEHYDRATION OF ORGANIC / WATER SOLUTIONS

(75) Inventors: Yu Huang, Palo Alto, CA (US); Jennifer Ly, San Jose, CA (US); Tiem Aldajani, San Jose, CA (US); Richard W. Baker, Palo Alto, CA (US)

(73) Assignee: MEMBRANE TECHNOLOGY AND RESEARCH, INC., Menlo Park, CA (US)

(21) Appl. No.: 13/191,014

(22) Filed: Jul. 26, 2011

Related U.S. Application Data
(63) Continuation-in-part of application No. 11/715,245, filed on Mar. 6, 2007, now Pat. No. 8,002,874.
Application No. 11/715,245 is a continuation-in-part of application No. 11/897,675, filed on Aug. 30, 2007.

(51) Int. Cl. C07C 29/76; B01D 71/38; C12P 7/06; C07C 45/78; C07C 51/42; B01D 71/06; B01D 71/12

(52) U.S. Cl. 435/161; 210/500.27; 210/500.42; 210/500.29; 568/917; 568/411; 562/608; 568/493

(57) ABSTRACT

Disclosed herein are processes for removing water from organic compounds, especially polar compounds such as alcohols. The processes include a membrane-based dehydration step, using a membrane that has a dioxole-based polymer selective layer or the like and a hydrophilic selective layer, and can operate even when the stream to be treated has a high water content, such as 10 wt % or more. The processes are particularly useful for dehydrating ethanol.

This application claims the benefit of U.S. application Ser. No. 11/715,245, filed Mar. 6, 2007, and U.S. application Ser. No. 11/897,675, filed Aug. 30, 2007, the disclosures of which are hereby incorporated herein by reference in their entireties.

This invention was made in part with Government support under award number NRCS-68-3A75-4-140, awarded by the United States Department of Agriculture. The Government has certain rights in this invention.

### FIELD OF THE INVENTION

The invention relates to the dehydration of organic/water solutions by means of separation membranes. The separation is performed under pervaporation conditions, in which the feed stream is in the liquid phase and the membrane permeate is in the vapor phase, or under vapor-phase conditions, in which the feed and permeate are in the vapor phase.

### BACKGROUND OF THE INVENTION

The production of fuel grade ethanol from renewable resources is expected to increase. Presently, many bioethanol plants in the U.S. use corn as the feedstock. Fermentation of lignocellulose to produce bioethanol is not currently economical. However, if research on this use of lignocellulose develops successfully, there will be an even larger increase in bioethanol production.

A major drawback to more economical use of bioethanol as a fuel is the energy used to grow the feedstock, to ferment it, and to separate a dry ethanol product from the fermentation broth. In this regard, the development of a lower energy ethanol separation (dehydration) process would be of considerable interest and use to bioethanol producers.

Dehydration of other organic liquids is also of economic importance. Isopropanol is widely used in the electronics industry and in the production of precision metal parts as a drying agent. The component to be dried is dipped or sprayed with anhydrous isopropanol, which removes any water, after which the component is dried. The isopropanol solvent eventually becomes contaminated with water and when it reaches about 10-30 wt % of water, it must be replaced. It would be economical to recover the isopropanol rather than disposing of it as a hazardous waste, as is presently done. Distillation of isopropanol/water is not economically feasible, as it forms an azeotrope at 87% isopropanol/13% water.

Another important organic liquid is acetic acid, the most widely used organic acid. Its primary industrial uses are for the production of vinyl acetate monomer and as a solvent in making terephthalic acid. In production of terephthalic acid, large aqueous acetic acid streams are produced, from which acetic acid must be recovered and a water stream produced that is sufficiently decontaminated to be properly discharged into the environment. An energy and cost-saving method for producing a dehydrated acetic acid stream suitable for recycling, along with a waste water stream suitable for discharge, would be of considerable economic interest.

While there are some commercially available membranes capable of dehydrating organic compounds by pervaporation, these membranes are hydrophilic, in that they swell significantly, or even dissolve, in an aqueous environment. They start to lose their separation properties, and are, therefore, unusable, even at water concentrations of just a few percent. The problem is exacerbated if the feed solution is hot. Unfortunately, many economically important organic solutions, such as those mentioned above, are not amenable to treatment by pervaporation for this reason.

There is thus a need in several industrial applications for more economical methods of dehydrating organic/water mixtures.

### SUMMARY OF THE DISCLOSURE

The invention is directed to processes for dehydrating organic/water solutions by vapor-phase or liquid-phase membrane separation.

In certain embodiments, the separation is carried out by running a feed stream of the organic/water solution across a membrane under pervaporation conditions. By pervaporation conditions, we mean that the vapor pressure of the desired faster permeating component is maintained at a lower level on the permeate side than on the feed side, and the pressure on the permeate side is such that the permeate is in the gas phase as it emerges from the membrane. These processes result, therefore, in permeate streams enriched in one component, in this case water, and residue liquid streams depleted in that component.

In other embodiments, the separation is carried out by running the feed stream across the membrane as a vapor, and by providing a difference in partial pressure between components on the feed and permeate sides. These processes again result in permeate vapor streams enriched in one component, in this case water, and residue vapor streams depleted in that component.

The membranes used in the processes of the invention have selective layers made from a hydrophobic fluorinated glassy polymer or copolymer. This polymer determines the membrane selectivity.

The polymer is characterized by having repeating units of a fluorinated, cyclic structure, the fluorinated ring having at least five members, where the fluorinated ring is preferably in the polymer backbone. Preferably, the polymer is formed from a monomer selected from the group consisting of fluorinated dioxoles, fluorinated dioxolanes, and fluorinated cyclically polymerizable alkyl ethers.

The polymer is further characterized by its hydrophobic nature. To be useful in the invention, the selective layer polymer should exhibit only modest swelling when exposed to significant concentrations of water, especially at high temperature.

The processes may be characterized in terms of having membrane selectivity of water to the organic compound of at least about 30, and a water permeance of at least about 500 gpu when challenged at 75° C. with a liquid mixture of 90 wt % ethanol/10 wt % water at a permeate pressure of less than 10 torr.

The fluorinated polymer is preferably heavily fluorinated, by which we mean having a fluorine:carbon ratio of atoms in the polymer of at least about 1:1. Most preferably, the polymer is perfluorinated.

In one embodiment, the dehydration process of the invention includes the following steps:

(a) providing a membrane having a feed side and a permeate side, the membrane having a selective layer comprising a polymer with a repeat unit of a hydrophobic fluorinated cyclic structure of an at least 5-member ring;

(b) passing a feed solution comprising at least 1 wt % water and a liquid organic compound across the feed side under pervaporation conditions;

(c) withdrawing from the feed side a dehydrated solution having a water content lower than that of the feed solution;

(d) withdrawing from the permeate side a permeate vapor having a higher water content than the feed solution.

In particular, the pervaporation conditions in step (b) may include providing the feed solution to the membrane at a temperature in the range of about 70° C. to 120° C.

In another embodiment the dehydration process includes the following steps:

(a) providing a membrane having a feed side and a permeate side, the membrane having a selective layer comprising a polymer with a repeat unit of a hydrophobic fluorinated cyclic structure of an at least 5-member ring;

(b) passing a feed vapor comprising at least 1 wt % water vapor and a vaporized organic compound across the feed side;

(c) providing a vapor pressure driving force for transmembrane permeation;

(d) withdrawing from the feed side a dehydrated vapor having a water content lower than that of the feed solution;

(e) withdrawing from the permeate side a permeate vapor enriched having a higher water content than the feed solution.

In particular, the water vapor and vaporized organic compound may be provided to the membrane in step (b) at a temperature in the range of about 70° C. to 130° C.

In any of the process embodiments disclosed herein, there may be further processing by passing at least a portion of a stream chosen from the permeate vapor and the dehydrated liquid or vapor stream to additional separation treatment. Any of the permeate or residue streams in the vapor phase may optionally be condensed. At least a portion of the permeate vapor is often condensed to provide or contribute to the driving force for transmembrane permeation.

Particularly preferred materials for the selective layer of the membrane used to carry out the processes of the invention are amorphous homopolymers of perfluorinated dioxoles, dioxolanes or cyclic alkyl ethers, or copolymers of these with tetrafluoroethylene. One class of preferred materials are copolymers having the structures:

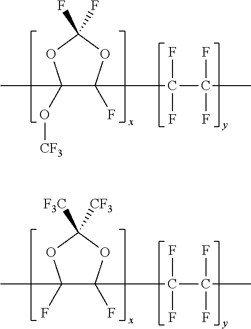

where x and y represent the relative proportions of the dioxole and the tetrafluoroethylene blocks, such that x+y=1.

A second class of preferred material has the structure:

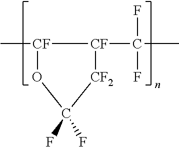

where n is a positive integer.

These preferred polymer materials are amorphous glassy materials with glass transition temperatures in the range of 100° C. to 250° C. The exceptional permeation properties of these membranes are derived from their structure. The materials are amorphous, glassy, highly fluorinated and without any ionic groups that would render the membranes hydrophilic or provide an affinity for other polar materials. As a result, they are not swollen to any significant extent by polar solvents, such as ethanol, isopropanol, butanol, acetone, acetic acid, and water. This low sorption, together with the intrinsic resistance to hydrolysis of fluoropolymers, makes these polymers chemically stable, even in hot organic/water mixtures that contain 20 wt % water or more, or are even predominantly aqueous.

These properties contrast with polymers, including crosslinked polyvinyl alcohol (PVA); polyvinylpyrrolidone (PVP); ion-exchange polymers, such as Nafion® and other sulfonated materials; and chitosan, that have previously been used for pervaporation membranes to remove small amounts of water from organic solutions.

We have found that membranes formed from fluorinated polymers as characterized above can operate satisfactorily as pervaporation membranes for dehydration of organic/water solutions. In other words, the membranes can be used to carry out dehydration under conditions in which the feed stream is essentially completely in the liquid phase, and hence the membrane is in continuous contact with liquid organic/water solutions throughout the duration of the dehydration process.

We have also found that membranes formed from fluorinated polymers as characterized above can operate satisfactorily as vapor-phase separation membranes for dehydration of organic/water solutions. In other words, the membranes can be used to carry out dehydration under conditions in which the feed stream is essentially completely in the vapor phase, and hence the membrane is in continuous contact with organic/water vapors throughout the duration of the dehydration process.

Because the preferred polymers are glassy and rigid, an unsupported film of the polymer may be usable in principle as a single-layer gas separation membrane. However, such a film will normally be far too thick to yield acceptable transmembrane flux, and in practice, the separation membrane usually comprises a very thin selective layer that forms part of a thicker structure, such as an asymmetric membrane or a composite membrane. Composite membranes are preferred.

The making of these types of membranes is well-known in the art. If the membrane is a composite membrane, the support layer may optionally be made from a fluorinated polymer also, making the membrane a totally fluorinated structure and enhancing chemical resistance. A useful support layer may comprise microporous polyvinylidene fluoride (PVDF). The membrane may take any form, such as hollow fiber, which may be potted in cylindrical bundles, or flat sheets, which may be mounted in plate-and-frame modules or formed into spiral-wound modules.

The driving force for transmembrane permeation is the difference between the vapor pressure of the feed liquid or vapor, and the vapor pressure on the permeate side. This pressure difference can be generated in a variety of ways, for example, by heating the feed liquid, compressing the feed vapor, and/or maintaining lower pressure or a partial vacuum on the permeate side.

The feed fluid to be treated by the processes of the invention contains at least water and an organic compound. The water may be a minor component or the major component of the fluid, and can be present in any concentration. The fluid may be a solution or a vapor-phase mixture.

The process embodiments of the invention can dehydrate water/organic solutions of any composition, from those that contain only small amounts of water, such as 1 wt % or less, to those that contain only small amounts of organics, such as 1 wt % or less. Embodiments of the invention are particularly useful for dehydrating organic solutions that contain more than 1 wt % water, such as 5 wt % water, 10 wt % water, 20 wt % water or more, which cannot be treated using conventional membranes.

The organic compound may be any compound or compounds able to form solutions or vapor mixtures with water. Our processes are particularly useful for removing water from polar organic compounds, such as ethanol and other alcohols, and other organic compounds in which water is readily soluble or miscible with water, such as esters or organic acids. Such separations are important in the manufacture of bioethanol and other biofuels.

The membranes and processes of the invention are particularly useful for dehydration of organic compounds such as alcohols, ketones, aldehydes, esters, or acids, in which water is readily soluble, or that are miscible with water over a wide concentration range. By readily soluble, it is meant that water has a solubility of at least about 10 wt % at room temperature and pressure. The invention is especially useful for dehydration of C₁ to C₆ alcohols, such as ethanol, isopropanol, and butanol.

The membrane separation processes may be configured in many possible ways, and may include a single membrane unit or an array of two or more units in series or cascade arrangements, as is familiar to those of skill in the art.

Another embodiment of the invention includes the following steps:

(a) providing a composite membrane having a feed side and a permeate side, the composite membrane comprising:
(i) a microporous support layer;
(ii) a first dense selective layer of a hydrophilic polymer; and
(iii) a second dense selective layer of a dioxole-based polymer having the structure

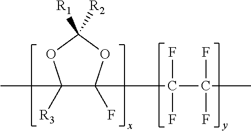

wherein R₁ and R₂ are fluorine or CF₃, R₃ is fluorine or —O—CF₃, and x and y represent the relative proportions of the dioxole and the tetrafluoroethylene blocks, such that x+y=1;

the first dense selective layer being positioned between the microporous support layer and the second dense selective layer;

(b) passing a feed solution comprising water and an organic compound across the feed side;

(c) withdrawing from the feed side a dehydrated solution having a lower water content than that of the feed solution;

(d) withdrawing from the permeate side a permeate vapor having a higher water content than that of the feed solution.

If the mixture is in the vapor phase, a basic embodiment of the above process includes the following steps:

(a) providing a composite membrane having a feed side and a permeate side, the membrane comprising:
(i) a microporous support layer;
(ii) a first dense selective layer of a hydrophilic polymer; and
(iii) a second dense selective layer of a dioxole-based polymer having the structure

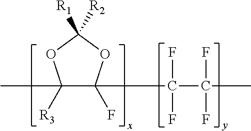

wherein R₁ and R₂ are fluorine or CF₃, R₃ is fluorine or —O—CF₃, and x and y represent the relative proportions of the dioxole and the tetrafluoroethylene blocks, such that x+y=1;

the first dense selective layer being positioned between the microporous support layer and the second dense selective layer;

(b) passing a feed vapor comprising water and an organic compound across the feed side;

(c) withdrawing from the feed side a dehydrated vapor having a water content lower than that of the feed solution;

(d) withdrawing from the permeate side a permeate vapor having a higher water content than the feed solution.

In both of the two process embodiments described above, the composite membrane has at least three layers: a microporous support layer; a thin, dense hydrophilic layer on the microporous support, and a thin, dense dioxole-based layer on the hydrophilic layer. Representative polymers that can be used for the hydrophilic layer include polyvinyl alcohol (PVA); cellulose acetate, and other cellulose derivatives; polyvinyl pyrrolidone (PVP); ion-exchange polymers, such as Nafion® and other sulfonated materials; and chitosan.

The dioxole-based layer is made from the specific dioxole-based polymers discussed with respect to the embodiments described above.

In the above embodiments, both the hydrophilic layer and the dioxole-based layer have selectivity for water over the organic compounds from which the water is to be removed. The intrinsic selectivity of the hydrophilic polymer is normally higher than that of the dioxole-based polymer.

Very surprisingly, we have found that, when membranes having the above structures are used, the processes of the invention can manifest higher selectivity for water over the organic compound than can be achieved under the same process conditions by either the hydrophilic polymer or the top layer polymer used alone as the selective layer of the membrane.

Another embodiment of the invention is a composite membrane comprising:

(a) a microporous support layer;

(b) a first dense selective layer of a hydrophilic polymer; and

(c) a second dense selective layer of a dioxole-based polymer having the structure

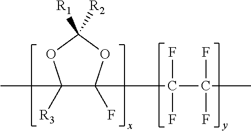

wherein R₁ and R₂ are fluorine or CF₃, R₃ is fluorine or —O—CF₃, and x and y represent the relative proportions of the dioxole and the tetrafluoroethylene blocks, such that x+y=1;

the first dense selective layer being positioned between the microporous support layer and the second dense selective layer.

The membrane is preferentially characterized in that, when challenged with a feed solution containing 20 wt % water at a set of operating conditions that include a temperature of 75° C., the composite membrane has a higher water/organic compound selectivity than that of either: (a) a first membrane having only a hydrophilic polymer selective layer of the same hydrophilic polymer as the first dense selective layer, or (b) a second membrane having only a dioxole-based polymer selective layer of the same dioxole-based polymer as the second dense selective layer, all as measured at the set of operating conditions.

The processes of the invention may include additional separation steps, carried out, for example, by adsorption, absorption, distillation, condensation or other types of membrane separation. One preferred embodiment of a process of this type comprises a stripping or distillation step, followed by a membrane separation step carried out using multi-layer composite membranes as described above.

In another aspect, the invention is a process for making ethanol by combining a fermentation step with multiple water/ethanol separation steps in series, one of the separation steps being a membrane dehydration step carried out using multi-layer composite membranes as described above.

Another, but less preferred, alternative is to use another type of perfluorinated, high-permeability material for the second selective layer.

It is to be understood that the above summary and the following detailed description are intended to explain and illustrate the invention without restricting it in scope.

### BRIEF DESCRIPTION OF THE DRAWINGS

FIG. 1 is an illustration of an embodiment of a membrane for use in accordance with the invention.

FIG. 2 is a schematic diagram of a system for dehydrating organic/water solutions in accordance with one process embodiment of the invention.

FIG. 3 is a schematic diagram of a system for dehydrating organic/water liquids in accordance with another embodiment of the invention.

FIG. 4 is a graph of the permeance of several liquids through Hyflon® AD 60 membrane as a function of critical volume.

FIG. 5 is a schematic drawing of a membrane for use in accordance with another embodiment of the invention.

FIG. 6 is a schematic drawing of a basic pervaporation process in accordance with one embodiment of the invention.

FIG. 7 is a schematic drawing of a basic vapor phase separation process in accordance with one embodiment of the invention, including compression of the feed vapor.

FIG. 8 is a schematic drawing of an embodiment of the invention in which membrane separation is combined with separation by stripping or distillation.

FIG. 9 is a schematic drawing of a process for producing alcohol from biomass.

FIG. 10 is a schematic drawing of an embodiment of the invention in which membrane separation is combined with stripping, and in which the membrane separation is performed as a two-step process.

FIG. 11 is a schematic drawing of a process for producing alcohol from biomass, in which the membrane separation is performed as a two-step process.

FIG. 12 is a graph comparing the performance of Hyflon®AD, Teflon®AF, and Celfa CMC VP-31 membranes in the form of a plot of permeate water concentration against feed water concentration at different feed water concentrations.

FIG. 13 is a plot comparing the water permeances of Celfa CMC VP-31 membranes having Hyflon®AD layers of different thicknesses.

FIG. 14 is a graph comparing the water/ethanol selectivity of Celfa CMC VP-31 membranes, Hyflon®AD membranes and Celfa CMC VP-31/Hyflon®AD membranes at different feed water concentrations.

FIG. 15 is a graph comparing water permeance of otherwise similar membranes having Hyflon®AD and Teflon®AF layers.

### DETAILED DESCRIPTION

The term gas as used herein means a gas or a vapor.

The terms hydrocarbon and organic vapor or organic compound are used interchangeably herein, and include, but are not limited to, saturated and unsaturated compounds of hydrogen and carbon atoms in straight chain, branched chain and cyclic configurations, including aromatic configurations, as well as compounds containing oxygen, nitrogen, halogen, or other atoms.

The term mixture as used herein means any combination of an organic compound and water, including solutions and vapor-phase mixtures. The term also refers to a solution, plus undissolved organics or water present as a separate phase. As used herein, the term mixture typically refers to mixtures of an organic compound and water that are liquid at room temperature and pressure.

The term separation factor refers to the overall separation factor achieved by the process. The separation factor is equal to the product of the separation achieved by evaporation of the liquid and the selectively achieved by selective permeation through the membrane.

The terms water/organic and organic/water solution and mixture used herein refer to mixtures of an organic compound and water that are liquid at room temperature and pressure.

All liquid mixture percentages herein are by weight unless otherwise stated. Gas or vapor mixture percentages are by volume unless otherwise stated.

The invention is a process for removing water from fluid mixtures containing water and organic compounds. The fluid may be in the gas or the liquid phase.

The separation is carried out by running a liquid or vapor stream of the water/organic mixture across a membrane that is selective for water to be separated over the organic component of the mixture. The process results, therefore, in a permeate stream enriched in water and a residue stream depleted of water, that is, dehydrated.

In certain embodiments, the process is performed under pervaporation conditions, as explained in more detail below, so that the feed is in the liquid phase and the permeate stream is in the gas or vapor phase.

In other embodiments, the process is performed in the gas phase so that the feed and permeate streams are both in the gas phase.

The process of the invention can be used to dehydrate many water/organic mixtures. We believe the process of the invention is of particular value in dehydrating solutions or vapor mixtures containing an organic compound that has good mutual miscibility or solubility with water, especially those containing an organic compound in which water has a solubility of at least about 5 wt % or 10 wt %. By way of example, the process of the invention is particularly useful for separating water from alcohols, ketones, aldehydes, organic acids and esters, including methanol, ethanol, isopropanol, butanol, acetone, acetic acid, and formaldehyde.

One or multiple organic compounds may be present in the solution to be dehydrated. A common example of a multi-organic mixture to be treated is ABE, an acetone-butanol-ethanol mixture typically produced by fermentation and used as a source of butanol and other valuable chemicals. The processes of the invention are characterized in terms of the materials used for the selective layers of the membrane, or by the process operating conditions in terms of water concentration in the feed mixture.

The streams to which the present invention applies are predominantly composed of organic components and water; however, inorganic components, including salts or dissolved gases, may be present in minor amounts.

Water may be a major or minor component of the mixture, and the water concentration may range from ppm levels to 80 wt % or more, for example. Unlike most prior art membrane dehydration processes, the process is suitable for streams containing large amounts of water, by which we mean streams containing more than about 10 wt % water, and in particular streams containing more than about 15 wt %, 20 wt %, 30 wt % water, or even streams in which water is the major component.

The scope of the invention is not limited to any particular type of stream. The feed streams may arise from diverse sources that include, but are not limited to, fermentation processes, chemical manufacturing, pharmaceutical manufacturing, electronic components manufacture, parts cleaning, processing of foodstuffs, and the like. As a particular example, the invention is useful for separating ethanol and water from a fermentation broth arising from bioethanol production.

In a first aspect of the invention, the selective layer of the membrane is made from a fluorinated glassy polymer, characterized by having repeating units of a cyclic structure, the ring having at least five members and being at least partially fluorinated. Generally, but not necessarily, the fluorinated ring is in the polymer backbone.

The ring structure within the repeat units may be aromatic or non-aromatic, and may contain other atoms than carbon, such as oxygen atoms.

In a second aspect, the process may be characterized by target separation characteristics. Preferably, the membranes provide a membrane selectivity of water to the organic compound of at least about 30, and a water permeance of at least about 500 gpu when challenged at 75° C. with a liquid mixture of 10 wt % water/90 wt % ethanol at a permeate pressure of less than 10 torr.

It should be understood that this characterization does not limit the process of the invention in this aspect to dehydration or to specific operating conditions. Membranes that meet this selectivity criterion may be operated at other temperatures and pressures.

It should further be understood that the definition relies on the selectivity, which is a membrane property, not the separation factor, which is a process attribute.

When characterized according to either aspect, the polymer is typically heavily fluorinated, by which we mean having a fluorine:carbon ratio of atoms in the polymer preferably of at least about 1:1, and more preferably is perfluorinated.

A measure of the chemically stable and hydrophobic nature of the polymer is its resistance to swelling when exposed to water. This may be measured in a very simple manner by weighing a film of the pure polymer, then immersing the film in boiling water for a period. When the film is removed from the water, it is weighed immediately, and again after the film has been allowed to dry out and reach a stable weight.

The selective layer of the membrane should be made from a polymer that is sufficiently stable in the presence of water that a film of the polymer immersed in water at 100° C. for 24 hours at atmospheric pressure will experience a weight change of no more than about 10 wt %, and more preferably, no more than about 5 wt %. If the film is removed from boiling water and weighed immediately, its weight will have increased compared with the original weight because of the presence of sorbed water. This weight increase should be no more than 10 wt %, and preferably, no more than 5 wt %. After the film has dried and the weight has stabilized, it is weighed again. If the film has suffered degradation as a result of the water exposure test, the weight may have decreased. The weight loss compared with the original weight should be no more than 10 wt %, and preferably, no more than 5 wt %.

Conventional materials used for dehydration membranes, including PVA, PVP, chitosan, and fluorinated ion-exchange materials, will typically fail this test, as will many materials that are insufficiently fluorinated or that do not have the defined ring structure.

Since the polymers used for the selective layer need to remain rigid and glassy during operation, they should have glass transition temperatures comfortably above temperatures to which they are typically exposed during the process. Polymers with glass transition temperature above about 100° C. are preferred, therefore, and, subject also to the other requirements and preferences above, the higher the glass transition temperature, in other words, the more rigid the polymer, the more preferred it is.

The polymers should preferably take amorphous, rather than crystalline form, because crystalline polymers are typically essentially insoluble and thus render membrane formation difficult, as well as exhibiting low gas permeability. The degree of crystallinity of the polymer should therefore normally be less than 50%, and preferably less than 20%, and even more preferably less than 10%.

Normally, and preferably, the polymer is non-ionic, that is, does not contain charged groups such as are incorporated into ion-exchange polymers. Polymers containing ionic groups are insufficiently stable in the presence of water, and fail the swellability test described above.

The selectivity of the membranes should be determined principally by the selective properties of the polymer. In other words, the polymer used for the selective layer should not contain any fillers, such as inorganic particles, that alter the polymer permeation properties. It is believed that the use of filled polymers, such as taught in U.S. Pat. No. 6,316,684, increases the free volume within the polymer and may raise the permeability of the polymer to very high levels, but reduce or eliminate the selectivity, as well as adversely affecting the mechanical stability of the membrane.

For similar reasons, materials having very high fractional free volume of greater than about 0.3 within the polymer itself are not preferred for at least some applications, especially if selectivity is important. In referring to fractional free volume (FFV), we mean the free volume per unit volume of the polymer, defined and calculated as:

FFV=SFV/vₛₚ

where SFV is the specific free volume, calculated as:

SFV=vₛₚ−v₀=vₛₚ1.3−v_{w}

and where:

vₛₚ is the specific volume (cm³/g) of the polymer determined from density or thermal expansion measurements,

v₀ is the zero point volume at 0° K, and

v_{w} is the van der Waals volume calculated using the group contribution method of Bondi, as known in the art.

Polymers with fractional free volume above 0.3 that should be avoided, at least for some applications, although they otherwise meet the criteria for suitable polymers, include perfluoro-2,2-dimethyl-1,3-dioxole copolymers (Teflon®AF polymers).

Preferred polymers for the selective layer of the membrane are formed from highly fluorinated monomers of (i) dioxoles, which are five-member rings of the form that polymerize by opening of the double bond in the ring, so that the ring forms part of the polymer backbone; or (ii) dioxolanes, similar five-member rings but without the double bond in the main ring; or (iii) polymerizable aliphatic structures having an alkyl ether group.

The polymers may be homopolymers of the repeating units of the fluorinated structures defined above. Optionally, they may be copolymers of such repeat units with other polymerizable repeat units. Preferably, these other repeat units should be fluorinated, or most preferably perfluorinated.

A number of suitable materials for use in such copolymers are known, for example, fluorinated ethers and ethylene. Particularly when perfluorinated, homopolymers made from these materials, such as polytetrafluoroethylene (PTFE) and the like, are very resistant to swelling by water. However, they tend to be crystalline or semi-crystalline and to have gas permeabilities too low for any useful separation application. As constituents of copolymers with the fluorinated ring structures defined above, however, they can produce materials that combine amorphous structure, good permeability, and good resistance to swelling by water. Copolymers that include tetrafluoroethylene units are particularly preferred.

Specific highly preferred materials include copolymers of tetrafluoroethylene with 2,2,4-trifluoro-5-trifluoromethoxy-1,3-dioxole having the structure:

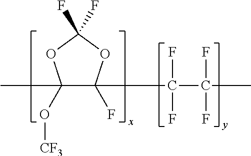

where x and y represent the relative proportions of the dioxole and the tetrafluoroethylene blocks, such that x+y=1.

Such materials are available commercially from Solvay Solexis, Inc., of Thorofare, N.J., under the trade name Hyflon®AD. Different grades are available varying in proportions of the dioxole and tetrafluoroethylene units, with fluorine:carbon ratios of between 1.5 and 2, depending on the mix of repeat units. For example, grade Hyflon®AD 60 contains a 60:40 ratio of dioxole to tetrafluoroethylene units, has a fractional free volume of 0.23 and a glass transition temperature of 121° C., and grade Hyflon®AD 80 contains an 80:20 ratio of dioxole to tetrafluoroethylene units, has a fractional free volume of 0.23 and a glass transition temperature of 134° C.

Other specific highly preferred materials include the set of polyperfluoro (alkenyl vinyl ethers) including polyperfluoro (allyl vinyl ether) and polyperfluoro (butenyl vinyl ether) that are cyclically polymerizable by the formation of repeat units of ether rings with five or six members in the ring.

A particular preferred material of this type has the structure:

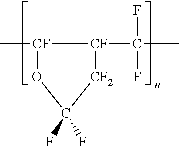

where n is a positive integer.

This material is available commercially from Asahi Glass Company, of Tokyo, Japan, under the trade name Cytop®. Cytop® has a fractional free volume of 0.21, a glass transition temperature of 108° C., and a fluorine:carbon ratio of 1.7.

A third group of materials that is believed to contain useful selective layer materials under some circumstances is:

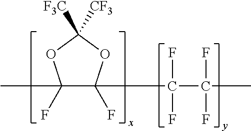

where x and y represent the relative proportions of the dioxole and the tetrafluoroethylene blocks, such that x+y=1.

Such materials are available commercially from DuPont of Wilmington, Del., under the tradename Teflon® AF.

The polymer chosen for the selective layer can be used to form films or membranes by any convenient technique known in the art, and may take diverse forms. Because the polymers are glassy and rigid, an unsupported film, tube, or fiber of the polymer is usable as a single-layer membrane.

Single-layer films will normally be too thick to yield acceptable transmembrane flux, however, and, in practice, the separation membrane usually comprises a very thin selective layer that forms part of a thicker structure, such as an integral asymmetric membrane or a composite membrane.

The preferred form is a composite membrane. Modern composite membranes typically comprise a highly permeable but relatively non-selective support membrane, which provides mechanical strength, coated with a thin selective layer of another material that is primarily responsible for the separation properties. Typically, but not necessarily, such a composite membrane is made by solution-casting the support membrane, then solution-coating the selective layer. Preparation techniques for making composite membranes of this type are well-known.

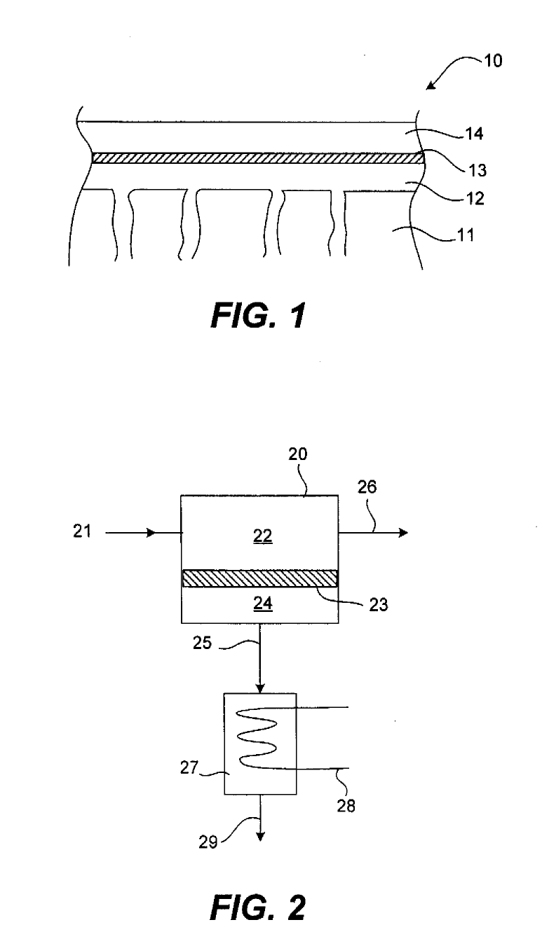

Referring to FIG. 1, if the membrane 10 is made in the form of a composite membrane, it is particularly preferred to use a fluorinated or perfluorinated polymer, such as polyvinylidene fluoride (PVDF), to make the microporous support layer 11. The most preferred support layers are those with an asymmetric structure, which provides a smooth, comparatively dense surface on which to coat the selective layer. Support layers are themselves frequently cast onto a backing web of paper or fabric.

The membrane 10 may also include additional layers, such as a gutter layer 12 between the microporous support layer 11 and the selective layer 13, or a sealing layer 14 on top of the selective layer 13. A gutter layer 12 generally has two purposes. The first is to coat the support with a material that seals small defects in the support surface, and itself provides a smooth, essentially defect-free surface onto which the selective layer 13 may be coated. The second is to provide a layer of highly permeable material that can channel permeating molecules to the relatively widely spaced pores in the support layer 11. Preferred materials for the gutter layer 12 are fluorinated or perfluorinated, to maintain high chemical resistance through the membrane structure, and of high permeability. A useful material for the gutter layer is Teflon® AF.

Such materials, or any others of good chemical resistance that provide protection for the selective layer 13 without contributing significant resistance to gas transport, are also suitable as sealing layers 14. The sealing layer 14 will typically be applied over the selective layer(s) 13 to provide protection of the selective layer. Silicone rubber is a useful material for the sealing layer 14.

Multiple selective layers 13 may also be used, as will be described in further detail below.

The thickness of the selective layer 13 or skin of the membranes can be chosen according to the proposed use, but will generally be no thicker than 10 μm, and typically no thicker than 5 μm. It is preferred that the selective layer be sufficiently thin that the membrane provide a pressure-normalized flux of the preferentially permeating component, as measured under the operating conditions of the process, of at least about 100 gpu (where 1 gpu=1×10⁻⁶ cm³(STP)/cm²·s·cmHg), more preferably at least about 500 gpu, and most preferably at least about 1,000 gpu.

It is preferred that the membranes provide a selectivity, as measured with the mixture to be separated and under normal process operating conditions, in favor of water, preferentially permeating component of the mixture, over the organic component from which it is to be separated of at least about 30, and more preferably at least about 50, at least about 100 or higher.

The separation factor provided by the process may be higher or lower than the membrane selectivity, depending on the volatilities of the organic component to be separated under the operating conditions of the process.

The membranes of the invention may be prepared in any known membrane form, such as flat sheets or hollow fibers, and housed in any convenient type of housing and separation unit. We prefer to prepare the membranes in flat-sheet form and to house them in spiral-wound modules. However, flat-sheet membranes may also be mounted in plate-and-frame modules or in any other way. If the membranes are prepared in the form of hollow fibers or tubes, they may be potted in cylindrical housings or otherwise as desired.

The membrane separation unit comprises one or more membrane modules. The number of membrane modules required will vary according to the volume flow of liquid to be treated, the composition of the feed liquid, the desired compositions of the permeate and residue streams, the operating temperature and pressure of the system, and the available membrane area per module.

Systems may contain as few as one membrane module or as many as several hundred or more. The modules may be housed individually in pressure vessels or multiple elements may be mounted together in a sealed housing of appropriate diameter and length.

One embodiment of apparatus useful for performing the process of the invention is shown in FIG. 2. Referring to this figure, a feedstream 21 comprising a liquid organic/water mixture, is passed into a membrane separation unit 20 and flows across the feed side 22 of membrane 23, which is characterized as described above. Under a vapor pressure difference between the feed 22 and permeate 24 sides of the membrane 23, water passes preferentially to the permeate side 24, and stream 25, enriched in water vapor, is withdrawn in the gas phase from the permeate side 24. The remaining liquid residue stream 26 is withdrawn from the feed side 22. The stream 25 may be condensed in condenser 27 cooled by line 28 containing a coolant to yield a liquid condensate stream 29. The residue stream 26 is withdrawn as the dehydrated product.

Transport through the membrane is induced by maintaining the vapor pressure on the permeate side of the membrane lower than the vapor pressure of the feed liquid. On the feed side of the membrane, the partial vapor pressure of any component will be the partial pressure of the vapor in equilibrium with the feed solution. Changing the hydrostatic pressure of the feed solution thus has a negligible effect on transmembrane flux or selectivity.

However, the vapor pressure on the feed side is a function of the temperature of the feed liquid. If the feed liquid emanates from an operation that is performed at elevated temperature, the feed liquid may already be hot, such as at 70° C., 80° C., or more. If the feed is at a temperature close to, or above, the glass transition temperature of the membrane material, it may be necessary to cool it. Thus, as a general guideline, feed temperatures above 130° C. are not preferred because of their effect on the module component and, sometimes, the membrane.

On the other hand, if the feed liquid is at a relatively low temperature, such as below about 25° C., it is often desirable to heat the feed liquid to increase the vapor pressure to attain pervaporation conditions, and hence the driving force for permeation. In general, the preferred range of feed temperatures is between about 70° C. and 120° C.

Although changing the hydrostatic pressure on the feed side has little effect, changing the permeate pressure has a major effect on transmembrane flux. The vapor pressure of a component on the permeate side can simply be maintained at atmospheric pressure, or even above atmospheric pressure, if desired. This mode of operation is preferred if the permeating component is to be recovered as a gas or vapor.

Alternatively, the vapor pressure on the permeate side can be reduced in several ways, for example, by drawing a vacuum on the permeate side of the membrane, by sweeping the permeate side to continuously remove permeating vapor, or by cooling the permeate vapor stream to induce condensation. Any such means may be used within the scope of the invention.

If the permeate is to be recovered in liquid form, it is possible simply to cool and condense the permeate stream, thereby generating a partial vacuum on the permeate side. Unless the vapor pressures on the feed side are particularly low (for example, if the feed components are thermally labile and the feed cannot be heated above ambient temperature), this will often suffice to generate adequate driving force, and avoid the cost and operational complexity of a vacuum pump.

Depending on the performance characteristics of the membrane, and the operating parameters of the system, the process can be designed for varying levels of separation. A single-stage pervaporation process typically removes up to about 90-95% of the water from the feed stream. This degree of separation is adequate for many applications.

If the residue stream requires further dehydration, it may be passed to a second bank of modules, after reheating if appropriate, for a second processing step. If the condensed permeate stream requires further concentration, it may be passed to a second bank of modules for a second-stage treatment. Such multi-stage or multi-step processes, and variants thereof, are familiar to those of skill in the art, who will appreciate that the process may be configured in many possible ways, including single-stage, multi-stage, multi-step, or more complicated arrays of two or more units in series or cascade arrangements.

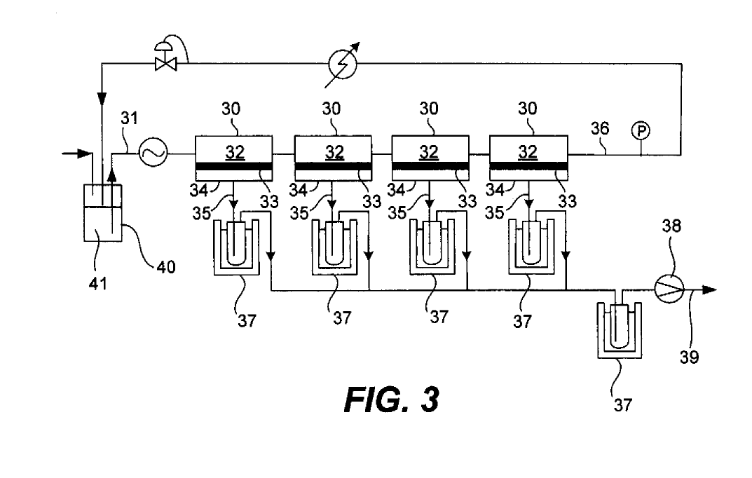

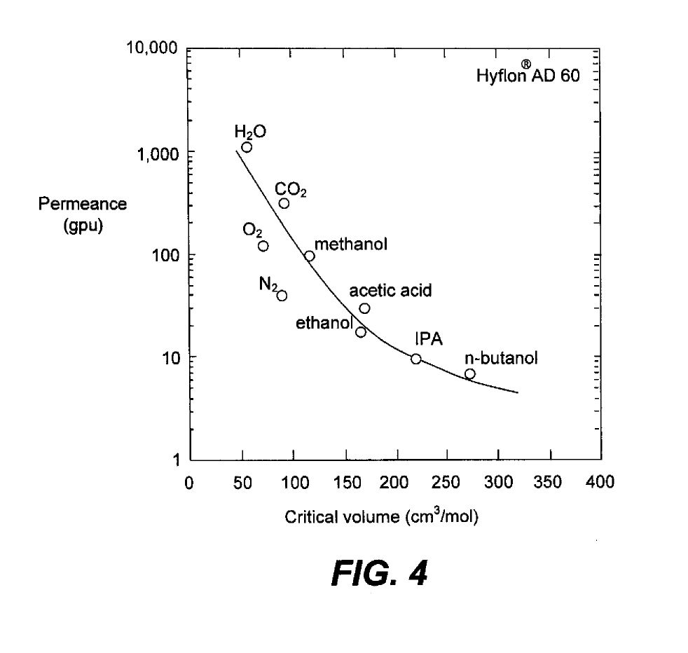

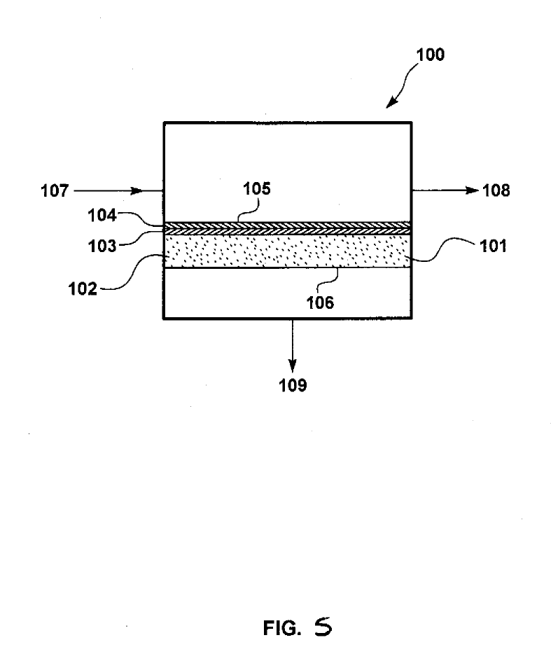

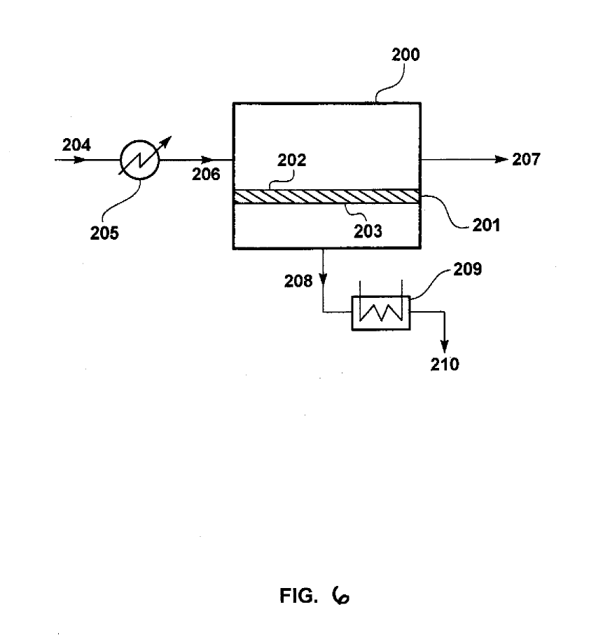

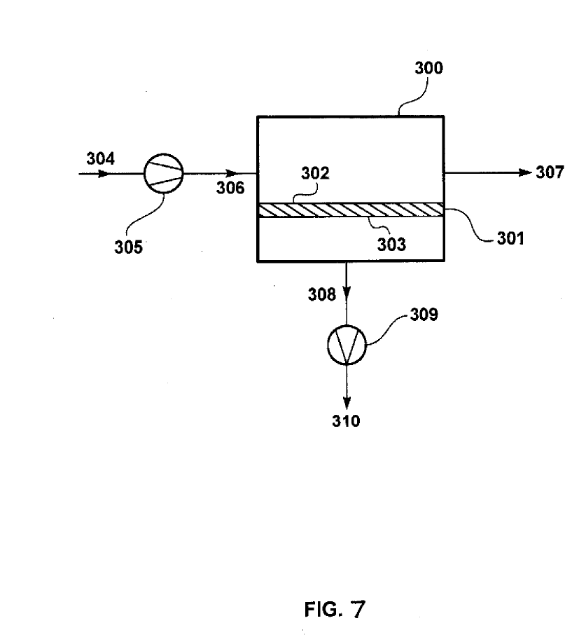

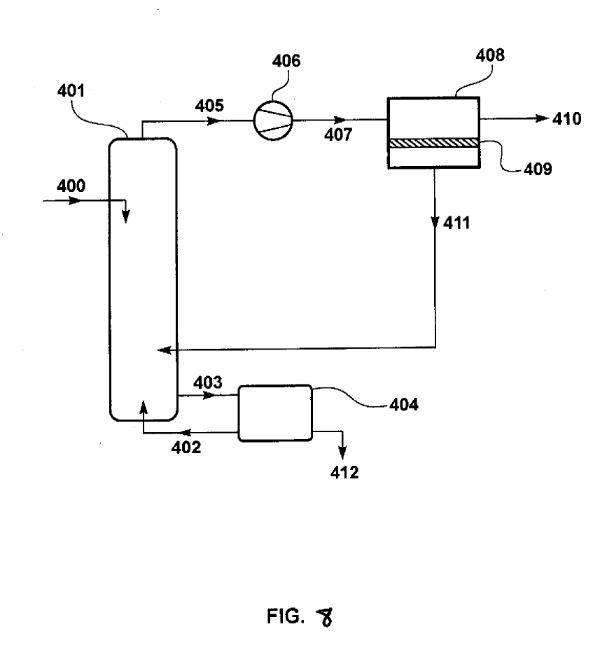

A system such as shown in FIG. 3 may be used to evaluate the performance of membrane samples or membrane modules in full recycle test mode as now described. Referring to this figure, a feedstream 31, comprising a liquid organic/water mixture 41, is passed from heated reservoir 40 into one or a plurality of membrane cells or membrane modules 30. The stream flows across the feed side 32 of membrane 33, which is characterized as described above. Under a vapor pressure difference between the feed 32 and permeate 34 sides of the membrane, water passes preferentially to the permeate side 34, and permeate vapor stream 35, enriched in water vapor, is withdrawn in the gas phase from the permeate side. Permeating water and organic compounds are condensed in cold traps 37. A vacuum pump 38 creates a vacuum on the permeate side of the membrane, and withdraws any uncondensed gases through line 39 that may have been dissolved in the feed solution. The condensed permeate collected over a time period is weighed and analyzed. The liquid residue stream 36 is withdrawn from the feed side 32 and recirculated to the feed reservoir 40. The desired test feed composition is maintained by adding fresh water to the reservoir through line 42

The measured fluxes and concentrations are converted to membrane permeances using the equations

$${J}_{i} = \frac{{P}_{i} ( {p}_{io} - {p}_{i l} )}{l} and {J}_{j} = \frac{{P}_{j} ( {p}_{jo} - {p}_{j l} )}{l}$$

where Jᵢ and Jⱼ are the water and organic component fluxes; Pᵢ and Pⱼ are the water and organic compound permeabilities; l is the membrane thickness; pᵢₒ and pⱼₒ are the feed side water and organic compound vapor pressures; and pᵢₗ and pⱼₗ (are the permeate side water and organic compound vapor pressures. Since the total permeate pressure (pᵢₗ+pⱼₗ) is less than 1 mm Hg, these two terms can be set to zero. The feed side partial pressures, pᵢₒ and pⱼₒ, are calculated using a process simulator (ChemCAD 5.5, Chemstations, Inc., Houston, Tex.) and an appropriate equation of state. In this way, the permeances Pᵢ/l of water and Pⱼ/l of organic compound can be calculated. The ratio of the permeances Pᵢ/l/Pⱼ/l gives the membrane selectivity α_{i/j}.

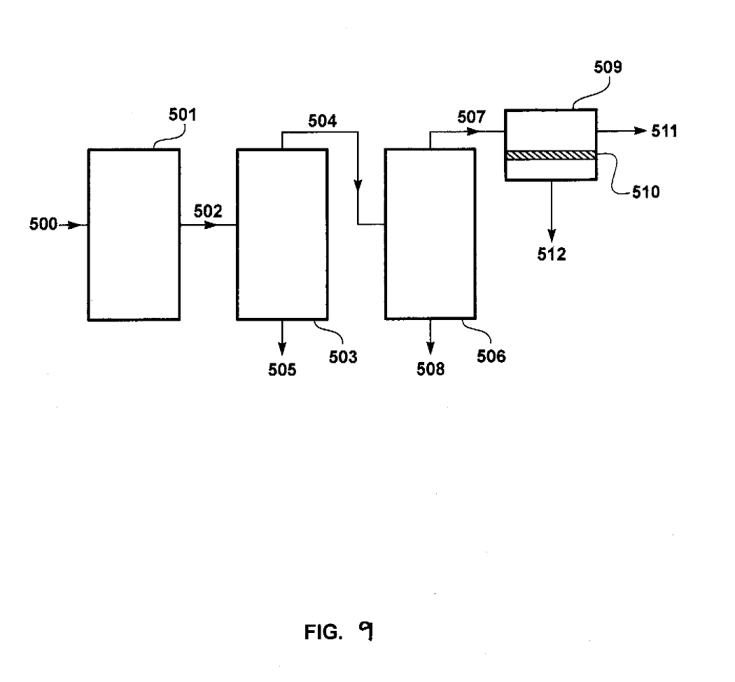

Representative results obtained with Hyflon®AD 60 perfluoro membranes are shown in Table 1. These results were obtained with large amounts of water in the feed solution. In all cases, the membranes were at least 50-fold more permeable to water than to the organic component. Some of the organic components could hardly be detected in the permeate, indicating a water/organic membrane selectivity of greater than 200. The permeance through a Hyflon®AD membrane decreases as the permeating component size increases, as illustrated in the plot shown in FIG. 4.

TABLE 1
Performance of Hyflon ®AD 60 Membranes with Feed
Solutions Containing Water as the Major Component

| | Feed Water | Permeate Water | Water | Organic Compound | |
|---|---|---|---|---|---|
| Organic | Concentration | Concentration | Permeance | Permeance | Selectivity |
| Compound | (wt %) | (wt %) | (gpu) | (gpu) | (water/organic) |
| Ethanol | 90.0 | 98.1 | 1,055 | 18 | 59 |
| Isopropanol | 89.6 | 99.9 | 1,166 | 10 | 117 |
| n-Butanol | 95.4 | 99.4 | 1,372 | 7 | 208 |
| Acetic acid | 90.8 | 99.8 | 1,945 | 30 | 65 |

An embodiment process of the invention whereby a liquid organic/water feed is supplied to the membrane includes the following steps:

(a) providing a membrane having a feed side and a permeate side, the membrane having a selective layer comprising a polymer with a repeat unit of a hydrophobic fluorinated cyclic structure of an at least 5-member ring;

(b) passing a feed solution comprising at least 1 wt % water and a liquid organic compound across the feed side under pervaporation conditions;

(c) withdrawing from the feed side a dehydrated solution having a water content lower than that of the feed solution;

(d) withdrawing from the permeate side a permeate vapor having a higher water content than the feed solution.

The dehydration process may also be performed in the vapor phase, where the feed is vaporized and passed through the membrane. In such a process, the permeate is collected as a vapor enriched in water vapor, and the retentate is collected as a dehydrated vapor. The residue and permeate vapors may optionally be condensed. The driving force for transmembrane permeation may be provided by applying a partial vacuum to the permeate side, pressurizing the feed side, or a combination of these techniques.

An embodiment process of the invention whereby an organic/water feed vapor is supplied to the membrane includes the following steps:

(a) providing a membrane having a feed side and a permeate side, the membrane having a selective layer comprising a polymer with a repeat unit of a hydrophobic fluorinated cyclic structure of an at least 5-member ring;

(b) passing a feed vapor comprising at least 1 wt % water vapor and a vaporized organic compound across the feed side;

(c) providing a vapor pressure driving force for transmembrane permeation;

(d) withdrawing from the feed side a dehydrated vapor having a water content lower than that of the feed solution;

(e) withdrawing from the permeate side a permeate vapor having a higher water content than the feed solution.

The apparatus design of FIG. 2 may also be used to carry out vapor separation processes. In this case, the feedstream 21 is at an elevated temperature, typically above 70° C., and is most preferably compressed to at least about 50 psia to provide a driving force for transmembrane permeation. The feed vapor passes into membrane separation unit 20 and flows across the feed side 22 of membrane 23, which is characterized as described above. Water vapor passes preferentially to the permeate side 24, and stream 25, enriched in water vapor, is withdrawn from the permeate side 24. The residue vapor stream 26 is withdrawn from the feed side 22, and may optionally be condensed. The driving force may be augmented by simply condensing the permeate stream 25 as shown in condenser 27, or by using a vacuum pump instead or as well on the permeate side.

It will often be preferred to either fully or partially condense the permeate vapor stream produced by the processes of the invention. Particularly when the separation is carried out in pervaporation mode, cooling and condensing the permeate will lower the vapor pressure on the permeate side of the membrane and facilitate transmembrane permeation.

Condensation may be carried out in any convenient manner, such as by heat exchange against an external coolant or a plant process stream, for example, as indicated by condenser 27 in FIG. 2. Optionally, the condensation step may be carried out using a dephlegmator, a partial condensation column from which the condensate leaves at the bottom and the uncondensed vapor leaves at the top. The dephlegmator tubes, fins or packing elements behave as wetted walls in which the up-flowing vapor and down-flowing condensate are in counter-current contact. This provides a separation improved, for example, four-fold or six-fold compared with that provided by simple condensation.

If a dephlegmation step has been used for other purposes, the processes of the invention can be used to dehydrate either the overhead or bottom stream from the dephlegmator. In fact, it is anticipated that the processes of the invention will often be useful in combination with other separation methods, such as distillation, absorption, or adsorption. It will be apparent to those of skill in the art that a pervaporation or vapor separation step in accordance with the invention may be used upstream or downstream of a distillation step, for example.

The membrane separation step may serve a variety of purposes. For example, it may lower the overall volume flow through the distillation column(s), thereby debottlenecking the plant, may provide energy and cost savings by reducing the reboiler duty or the reflux ratio, or may break an azeotrope, rendering one or both of the residue and permeate streams amenable to distillation.

For example, if the overhead stream is such that an azeotrope is formed, the overhead can be condensed, and the condensate subjected to pervaporation, to break the azeotrope. The residue or permeate stream, depending on the nature of the separation, may be withdrawn as a purified product stream, and the other stream may be returned to the appropriate position in the column.

Likewise, the membrane separation step can be used to treat the bottom stream from the distillation column, with the residue or permeate stream forming the purified product, and the other stream being returned to the column. A side cut from the column can also be treated.

Alternative embodiments of the invention are shown in FIGS. 5-9. A basic flow diagram of this embodiment of the invention is shown in FIG. 5. Referring to this figure, feed stream, 107, enters membrane unit, 100, and flows across the feed side, 105, of composite membrane, 101. The membrane in this embodiment has three layers: a microporous support membrane, 102; a hydrophilic layer, 103; and a dioxole-based layer, 104. The hydrophilic layer is positioned between the support layer and the dioxole-based layer. These three layers are now discussed individually.

So long as it offers essentially no resistance to permeation compared with the selective layers, the nature of the support membrane is not critical to the invention, and the membrane may be made from such typical known materials as polysulfone, polyetherimide (PEI), polyacrylonitrile, and polyvinylidene fluoride (PVDF), for example. The most preferred support layers are those with an asymmetric structure, having a smooth, comparatively dense surface on which to coat the selective layer. Optionally and preferably, the support membrane includes a porous backing web, not shown, onto which the support membrane has been solution-cast.

The hydrophilic polymer layer is adjacent to the support membrane, and the dioxole-based polymer layer is the top selective layer. The layers operate together to provide properties that could not be provided by either layer alone. Both layers are made from polymers that have high water/organic compound selectivity, at least when tested with solutions that contain no more than about 10 wt % water. The hydrophilic polymer has higher intrinsic selectivity than the dioxole-based polymer, and preferably should have a selectivity of at least about 200 under low water concentration test conditions (less than 10 wt % water). Suitable hydrophilic polymers include, but are not limited to, polyvinyl alcohol (PVA); cellulose acetate and all other cellulose derivatives, polyvinyl pyrrolidone (PVP); ion-exchange polymers, such as Nafion® and other sulfonated materials; and chitosan.

The top selective layer polymer is a dioxole-based polymer, as described above with respect to previous embodiments of the invention. As discussed above, these polymers are hydrophobic, and generally exhibit much lower water permeability and water/organic compound selectivity than hydrophilic polymers membranes under low water concentration test conditions (less than 10 wt % water). Despite their hydrophobic nature, however, we previously discovered that membranes formed from these polymers can operate well to dehydrate organic/water solutions. Unlike their hydrophilic counterparts, they can maintain a relatively stable performance when exposed to fluid mixtures with high water concentrations, such as more than 20 wt % water, even when the mixture is hot.

A measure of the chemical stability and hydrophobic nature of the polymer is its resistance to swelling when exposed to water. This may be measured in a very simple manner by weighing a film of the pure polymer, then immersing the film in boiling water for a period. When the film is removed from the water, it is weighed immediately, and again after the film has been allowed to dry out and reach a stable weight.

As discussed above, the dioxole-based polymer that forms the top selective layer of our membrane is sufficiently stable in the presence of water that a film of the polymer immersed in water at 100° C. for 24 hours at atmospheric pressure will experience a weight change of no more than about 10 wt %, and more preferably, no more than about 5 wt %. If the film is removed from boiling water and weighed immediately, its weight will have increased compared with the original weight because of the presence of sorbed water. This weight increase should be no more than 10 wt %, and preferably, no more than 5 wt %. After the film has dried and the weight has stabilized, it is weighed again. If the film has suffered degradation as a result of the water exposure test, the weight may have decreased. The weight loss compared with the original weight should be no more than 10 wt %, and preferably, no more than 5 wt %.

By contrast, the polymer used for the hydrophilic layer almost always fails this test.

The preferred dioxole-based polymers for use in this embodiment of the invention are copolymers having the structure:

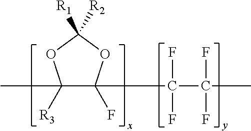

wherein R₁ and R₂ are fluorine or CF₃, R₃ is fluorine or —O—CF₃, and x and y represent the relative proportions of the dioxole and the tetrafluoroethylene blocks, such that x+y=1.

Specific highly preferred materials include copolymers of tetrafluoroethylene with 2,2,4-trifluoro-5-trifluoromethoxy-1,3-dioxole having the structure:

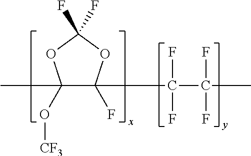

where x and y represent the relative proportions of the dioxole and the tetrafluoroethylene blocks, such that x+y=1.

Such materials are available commercially from Solvay Solexis, of Thorofare, N.J., under the trade name Hyflon®AD. Different grades are available varying in proportions of the dioxole and tetrafluoroethylene units, with fluorine:carbon ratios of between 1.5 and 2, depending on the mix of repeat units. For example, Hyflon®AD60 contains a 60:40 ratio of dioxole to tetrafluoroethylene units; Hyflon®AD80 contains an 80:20 ratio of dioxole to tetrafluoroethylene units.

Yet other preferred materials have the structure:

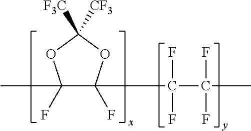

where x and y represent the relative proportions of the dioxole and the tetrafluoroethylene blocks, such that x+y=1. Such materials are available commercially from DuPont Fluoroproducts of Wilmington, Del., under the trade name Teflon®AF. These materials are also available in different grades of different glass transition temperature. Teflon®AF1600 is our most preferred grade for this embodiment of the invention.

As discussed above, the preparation of composite membranes for gas and liquid separations is well-known in the art, and the membrane may be made by any convenient technique. Typically, the microporous support membrane is cast from solution onto a removable or non-removable backing, and the selective layers are solution coated onto the support. As mentioned above, it is preferred that the support membrane have an asymmetric structure, with much finer, smaller pores in the skin layer to facilitate coating. Such membranes may be made by the Loeb-Sourirajan process.

The hydrophilic selective layer is positioned between the support membrane and the top selective layer. The hydrophilic layer may be contiguous with the support membrane. In this case, the hydrophilic layer is usually deposited directly on the support surface by solution coating, followed by curing, cross-linking, or any other post-deposition treatment that may be needed. Such steps are familiar to those of skill in the art.

As a less preferred alternative, the support membrane may be cast as an integral asymmetric membrane from a suitable hydrophilic polymer, the casting recipe and technique being such that the skin layer of the asymmetric membrane is sufficiently dense, and hence selective, to serve as the hydrophilic layer. Membranes having a cellulose triacetate hydrophilic selective layer can be made in this way, for example.

Instead of the support and hydrophilic layers being contiguous, a gutter layer may optionally be used between the support membrane and the hydrophilic selective layer, for example, to smooth the support surface and channel fluid to the support membrane pores. In this case, the support membrane is first coated with the gutter layer, then with the hydrophilic layer.

The dioxole-based selective layer is applied as the top selective layer, usually directly onto the hydrophilic layer, by solution coating. Optionally, a sealing layer may be applied on top of the dioxole-based layer to protect the membrane. The use of highly permeable polymers as sealing or gutter layers is known in the art.

The membranes may be made in the form of flat sheets or hollow fibers, for example, and formed into membrane modules of any convenient type. We prefer to use flat sheet membranes assembled into spiral-wound modules.

The hydrophilic layer is shielded from direct contact with the feed fluid by the dioxole-based top selective layer. We have discovered that this prevents the hydrophilic layer from excessive swelling and degradation in the presence of liquids or vapors of high water concentration. As a result, the embodiment processes of the invention provide higher selectivity under certain operating conditions than prior art processes using membranes with only a hydrophilic selective layer.

As a guideline, the membranes should preferably provide a selectivity of at least about 50 and more preferably at least about 100, when tested with a 50/50 ethanol/water mixture at 75° C.

We have found, very surprisingly, that the membranes of this embodiment of the invention offer higher selectivity, under conditions where they are exposed to a high water concentration in the feed, than can be achieved either by a membrane having only a hydrophilic selective layer or a membrane having only a dioxole-based selective layer under the same set of operating conditions. Comparative test results demonstrating this unexpected phenomenon with feed solutions containing 20 wt % water or more, and carried out at the high temperature of 75° C. are given in Example 7.

The thickness of each of the selective layers independently should generally be no thicker than 10 μm, and preferably no thicker than 5 μm. In particular, it is preferred that the dioxole-based layer be very thin, such as less than 2 μm, as the dioxole is the less permeable polymer, and an overly thick layer will reduce the permeance of the membrane to an undesirably low level. Most preferably, the dioxole-based selective layer thickness should be in the range 0.1-1 μm.

Preferably, the finished membrane provides a water permeance of at least about 500 gpu, and most preferably at least about 1,000 gpu, coupled with a water/organic compound selectivity of at least about 100, when in operation in the processes of the invention.

The separation factor provided by the process may be higher or lower than the membrane selectivity, depending on the relative volatilities of the organic component and water.

Returning to FIG. 5, feed stream 107 is passed across feed side 105 of water-selective membrane 101. The feed stream is separated into residue stream, 108, which is withdrawn from the feed side as a water-depleted residue, and permeate stream, 109, which is withdrawn from the permeate side, 106, as a water-enriched permeate, 109.

The driving force for transmembrane permeation of water is the difference between the water vapor pressure on the feed and permeate sides. In other words, the vapor pressure of water on the feed side is higher than the vapor pressure on the permeate side. This pressure difference can be generated in any convenient manner, such as by heating or compressing the feed stream, by maintaining the permeate side under vacuum, or by a combination of these methods.

The preferred method of generating driving force depends to some extent on whether the process is to be performed in pervaporation or vapor separation mode. In pervaporation mode, the feed is in the liquid phase, and the pressure on the permeate side is such that the permeating water is in the gas phase as it emerges from the membrane. In vapor permeation mode, the feed, residue, and permeate streams are all vapors as they enter and leave the membrane unit.

A basic representative embodiment of the invention in pervaporation mode is shown in FIG. 6. In this embodiment, it is assumed that the transmembrane driving force is created by heating the feed solution and by condensing the permeate vapor. Other methods of providing the driving force, such as by using a vacuum pump on the permeate side, could optionally be used.

Referring to this figure, liquid feed solution, 204, is heated in step, 205, and enters membrane unit or step, 200, as heated feed solution, 206. The membrane unit contains water-selective composite membrane, 201, of the composite type described above, having feed side, 202, and permeate side, 203. Water preferentially permeates the membrane and emerges from the permeate side as permeate vapor stream, 208. This stream is passed into condenser or condensation step, 209, and is withdrawn as water-rich condensate stream, 210. Condensation of the permeate reduces the vapor pressure in the permeate lines, thereby exposing the permeate side of the membrane to a partial vacuum and increasing the transmembrane driving force. The dehydrated residue solution is withdrawn as stream 207 from the feed side.

A basic representative embodiment of the invention in vapor separation mode is shown in FIG. 7. In this embodiment, it is assumed that the transmembrane driving force is created by compressing the feed vapor and using a vacuum pump to create a partial vacuum on the permeate side. Other methods of providing the driving force, such as condensing the permeate vapor, could optionally be used.

Referring to this figure, feed vapor, 304, is compressed in compressor or compression step, 305, and enters membrane unit or step, 300, as compressed feed vapor, 306. The membrane unit contains water-selective composite membrane, 301, of the composite type described above, having feed side, 302, and permeate side, 303. Water vapor preferentially permeates the membrane and emerges from the permeate side as permeate vapor stream, 308. This vapor is drawn through vacuum pump, 309, and exhausted as water-rich vapor stream, 310. The dehydrated residue vapor is withdrawn as residue stream, 307, from the feed side.

In both the pervaporation and vapor separation modes of operation, supplying the feed stream to the membrane at elevated temperature increases the transmembrane driving force and is preferred. Most preferably, the feed stream temperature should be in the range of 30° C. to 120° C., such as 40° C., 60° C., 75° C., or 100° C., depending on the specific separation to be performed and other operating parameters. For example, for ethanol/water separations, a typical feed stream temperature might be 75° C., 90° C., or 110° C. Temperatures much above 130° C. are not preferred, and temperatures above about 140° C. should be avoided, because of potential damage to the polymeric membranes or other module components, such as glues and spacers.

In the simple schematic diagrams of FIGS. 5-7, the membrane separation step is indicated as single box 100, 200, or 300. In each case, this step is carried out in a membrane separation unit that contains one or more membrane modules. The number of membrane modules required will vary according to the volume flow of the stream to be treated, the composition of the stream, the desired compositions of the permeate and residue streams, the operating temperature and pressure of the system, and the available membrane area per module.

Systems may contain as few as one membrane module or as many as several hundred or more. The modules may be housed individually in pressure vessels, or multiple elements may be mounted together in a sealed housing of appropriate diameter and length. Most preferably, the membrane modules, also known as membrane elements, are housed in a vessel that provides heating or reheating within the vessel, as disclosed in U.S. Pat. No. 7,758,754.

Depending on the performance characteristics of the membrane, and the operating parameters of the system, the process can be designed for varying levels of separation. A single-stage process in a typical example of a feed containing 20 wt % water might remove about 90% of water from the feed stream, to yield a residue stream containing 2 wt % water and a permeate stream containing 70 or 80 wt % water. This degree of separation is adequate for many applications.

If the residue stream requires further purification, it may be passed to a second bank of modules, after reheating if appropriate, for a second processing step. This is generally referred to as a two-step process. If the permeate stream requires further concentration (to recapture a valuable organic that might otherwise be lost, for example), it may be passed to a second bank of modules for a second-stage treatment. This is generally referred to as a two-stage process. Such multi-stage or multi-step processes, and variants thereof, are familiar to those of skill in the art, who will appreciate that the process may be configured in many possible ways, including single-stage, two-step, two-stage, or more complicated arrays of two or more units in series or cascade arrangements.

The dehydrated organic compound residue stream withdrawn from the membrane separation step is usually the primary product of the process and may pass to any destination. In most dehydration operations, it is preferred to configure the membrane separation steps to achieve a dehydrated product that contains less than 10 wt % water. Depending on the specific separation, much lower water concentrations in the product, such as less than 5 wt %, less than 1 wt %, or less than 0.5 wt % water, may be required.

The water-rich permeate stream may be sent to any destination. Often, but not necessarily, this stream is simply a waste stream that is clean enough, as a result of the process of the invention, to discharge to the local sewer system. In other circumstances, it may be useful to recirculate this relatively clean water stream within the process, or to the plant that produced the feed stream.

The processes of the invention may also include additional separation steps, carried out, for example, by adsorption, absorption, distillation, condensation, or other types of membrane separation, either before or after the membrane separation process that has been described above.

One example of such a process is shown in FIG. 8, which is a schematic drawing of an embodiment of the invention in which membrane separation is combined with stripping or distillation. The figure is described below as it relates to the removal of water from a stream exiting a fermenter used to produce ethanol. This description is not intended to be limiting—it will be apparent to those of skill in the art that the same or a similar process could be applied to separate other organic compounds, of any type and from any source, that have suitable volatility to be steam stripped preferentially into the overhead vapor.

Referring to FIG. 8, feed stream 400 is a liquid stream from a fermentation process, containing ethanol and water, the ethanol being the minor component. For example, the ethanol content of the stream might be 3 wt %, 5 wt %, 10 wt %, or 12 wt %. In the case that the feed derives directly from a fermenter, the stream may also contain other material that has been carried over from the fermentation step, including solid matter such as cell remnants and insoluble cellulosic matter, as well as sugars, proteins, or the like.

The stream enters stripping column, 401. Such columns are well-known and used in many industrial applications. The column may be of any design that allows contact between liquid and vapor phases in the column, and is preferably a packed or plate column. Pressure and temperature conditions within the column may be adjusted, as is known in the art, to suit the specific separation that is being carried out.

In the representative ethanol/water separation example of FIG. 8, the column is often referred to as the beer still. The beer still performs a stripping function, the stripping vapor being provided by a reboiler at the base of the column, but has no rectifier section. This column is typically, but not necessarily, operated under partial vacuum conditions, which can be set by the suction pressure of compressor, 406. If the feed stream is introduced to the column directly from the fermentation step, it will typically be at about 30-40° C.

As the feed liquid descends the column, it is contacted with a rising flow, 402, of stripping vapor generated by reboiler, 404, at the base of the column. Ethanol is transferred preferentially over water into the rising vapor phase, producing an ethanol-enriched vapor stream, 405, that is withdrawn from the top of the column. In the representative embodiment shown in FIG. 4, this vapor stream typically contains about 50 wt % each of water and ethanol.

Bottoms stream, 403, leaves the bottom of the stripper column, and will usually pass through the reboiler before being withdrawn as discharge stream, 412. This stream contains water and any solids that have been carried into the column with the feed stream, but typically contains less than 1 wt % ethanol, and preferably, 0.1 wt % ethanol or less. This stream may be returned to the fermenter, discharged, concentrated to recover the contained solids, or otherwise disposed of as appropriate.

The overhead stream from the column passes through compressor 406, emerging as compressed vapor stream, 407, and enters the membrane separation unit, 408, which contains water-selective composite membranes, 409, of the composite type described above. As with FIGS. 5-7, the membrane separation unit contains one or multiple membrane modules, arranged in one or multiple steps or stages. For example, the configuration may involve two membrane sub-steps, with the residue stream from the first sub-step being passed as feed to the second sub-step.

Water preferentially permeates the membrane and emerges from the permeate side as permeate vapor stream, 411. This vapor may be returned to the column to augment the stripping vapor from the reboiler. The dehydrated residue vapor is withdrawn as residue stream, 410, from the feed side.

The invention is expected to be particularly beneficial in the production of biofuels, that is, fuels produced from biomass of some type. FIG. 9 illustrates this aspect of the invention and, like FIG. 8, is described as it relates to the production of ethanol, although it is not so limited, and could be used to produce other alcohols or alcohol mixtures, for example.

Referring to FIG. 9, feed biomass, 500, enters fermentation plant or step, 501. The biomass feedstock may be any biomass that contains a fermentable sugar, or that can be processed to produce a fermentable sugar. Examples of biomass that contains fermentable sugars include corn, sugar cane, beets, fruits and vegetables, wastes from processing fruits and vegetables, and cheese whey. Examples of wastes that can be processed to make fermentable sugars include cellulosic materials, such as grasses, grain stalks, hulls, and other agricultural wastes, and lignocellulosic materials, such as woody materials and wood wastes.

The fermentation itself uses any reaction that can convert a sugar to an alcohol, and may be carried out in any convenient manner. Numerous fermentation techniques appropriate for use in alcohol production are well-known in the art and described in the literature. The reactor may take the form of a single vessel, or may be staged, for example, to provide different fermentation conditions in each stage. The reactor may be operated in any mode, such as batch, fed-batch, semi-continuous, or continuous mode.

If the source material itself does not contain adequate quantities of sugar, but may be treated to form sugars, the fermentation step may include sub-steps that convert starch or cellulose to sugar, or that break down lignin and then convert exposed cellulose. These steps may be carried out as pre-treatment before the material enters the fermentation vessel, or may be performed simultaneously with the fermentation.

The fermentation step may also include one or more filtration steps, to treat the fermentation broth to recover yeast cells or nutrients, or to remove suspended solids or dissolved salts, for example.

The product broth or solution from the fermentation step, 502, consists of water, ethanol as a minor component and, typically, at least some other dissolved or suspended matter. The ethanol concentration in this stream is usually, but not necessarily, less than 15 wt % ethanol, such as 5 wt %, 10 wt %, or 12 wt % ethanol. This stream passes to first separation step, 503. This step removes some of the water, and raises the ethanol concentration by at least about three-fold or five-fold, and preferably to at least about 50 wt %. The step may be carried out in a beer still, as described above with respect to the process embodiment depicted in FIG. 8, or by any other separation technique capable of raising the ethanol concentration sufficiently. In addition to the conventional beer still, another preferred option is to use membrane separation for this step. In this case, the membranes to be used will preferably be selective in favor of ethanol over water, so as to create an ethanol-enriched permeate stream and a residue stream that is mostly water. The configuration and use of such membranes is taught in U.S. Pat. Nos. 6,755,975 and 7,732,173.

This step produces an ethanol-enriched stream, 504, and an ethanol-depleted, water-rich stream, 505. Preferably, this stream contains less than 1 wt % ethanol, as can be achieved with either a stripping column or a membrane separation unit.

The ethanol-rich stream, which may be in the vapor or liquid phase, passes to second separation step, 506. The goal of this step is to dehydrate the ethanol to produce a product that preferably contains at least 90 wt %, and more preferably higher, such as 95 wt % ethanol or above. The step may be carried out using any separation technique capable of raising the ethanol concentration to the desired level. In existing processes that do not incorporate a membrane separation step, this step is usually carried out by distillation. In this case, the maximum ethanol concentration of the ethanol-rich overhead stream will be the azeotropic concentration, that is, 96 wt % ethanol/4 wt % water. As another example, the step may be carried out by dephlegmation, as described in U.S. Pat. No. 6,755,975.

The second separation step produces ethanol-rich stream, 507, and ethanol-lean stream, 508. This water-enriched, ethanol-depleted stream may optionally be returned to the inlet of the first separation step.

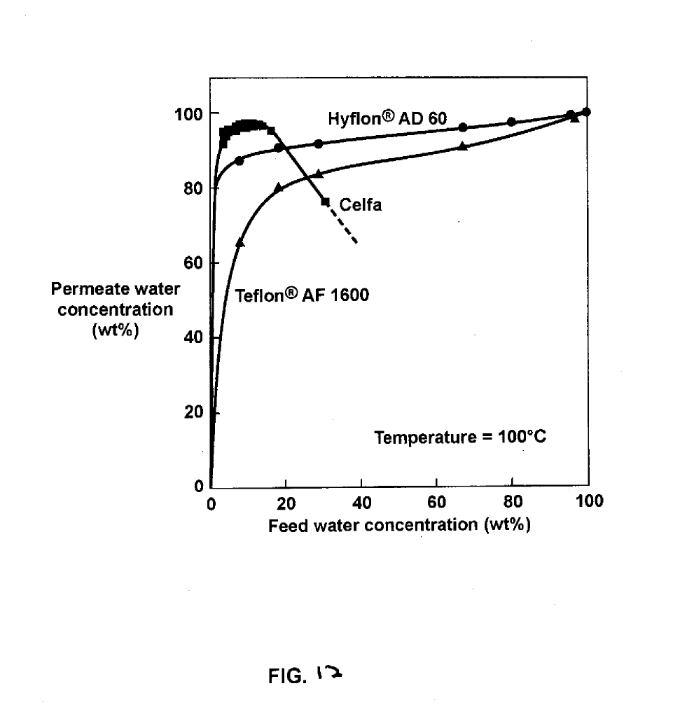

The ethanol-rich stream, preferably containing at least 90 wt % ethanol, is passed as vapor or liquid to membrane dehydration unit or step, 509. This step uses one or multiple membrane modules containing water-selective membranes, 510, of the composite type described above. The modules are arranged in one or multiple steps or stages. Performing this step as two sub-steps, as shown in FIG. 11, discussed in the Examples section, is often advantageous.

Water preferentially permeates the membranes, to produce a dehydrated ethanol product as the residue stream and a water-enriched permeate vapor stream, 512. The permeate vapor stream may optionally be recirculated within the process. The dehydrated ethanol product should preferably contain at least 99 wt % ethanol, and more preferably, at least 99.5 wt % or 99.7 wt % ethanol.

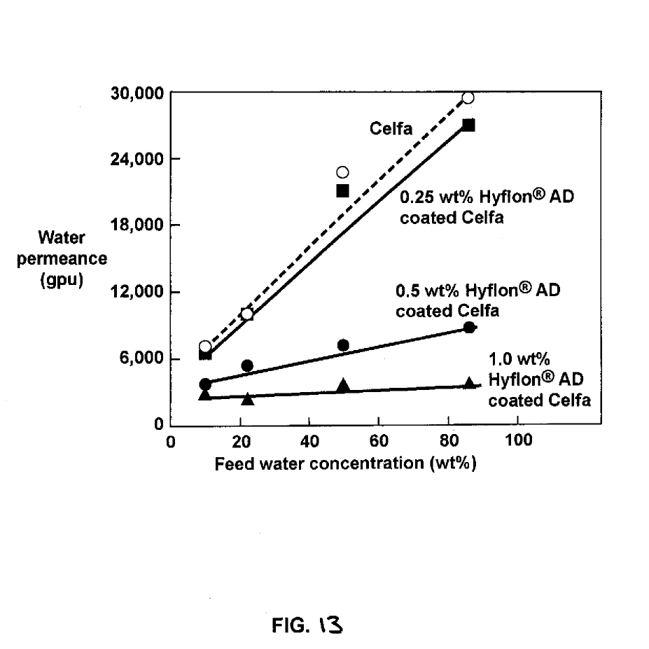

As a less preferred alternative in these embodiments of the invention, a different type of polymer material may be used for the second selective layer. This material should be capable of deposition as a very thin, dense, non-porous layer onto the hydrophilic selective layer, should be insoluble in water, and exhibit little or no swellability in water, so as to provide stable water permeation results at least comparable with those shown in FIGS. 12-14, and discussed in the Examples section below. The material should also exhibit water/ethanol selectivity of at least about 30, and be of sufficiently high permeability that the finished membrane has a water permeance of at least about 500 gpu.

One example of such a less preferred material is a perfluorinated cyclic alkyl ether having the structure:

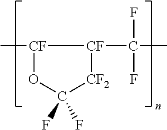

where n is a positive integer.

This material is available commercially from Asahi Glass Company, of Tokyo, Japan under the trade name Cytop®.

The invention is now further described by the following examples, which are intended to be illustrative of the invention, but are not intended to limit the scope or underlying principles in any way.

### EXAMPLES

### Example 1

### Membranes

Composite membranes were made. All of them included microporous support layers made using standard casting procedures to apply polyvinylidene fluoride (PVDF) solution to polyphenyl sulfide (PPS) paper. One set of membranes had a Hyflon®AD60 selective layer applied from a 0.5 wt % solution; the other had a Teflon®AF1600 selective layer applied from a 1 wt % solution.

Celfa CMC VP-31 composite membrane was purchased from Folex-Celfa AG, Bahnhofstrasse 6423, Seewen, Switzerland. The membrane is a composite membrane suitable for pervaporation, with a hydrophilic selective layer of unknown composition.

The Celfa CMC VP-31 has only a hydrophilic selective layer; the membranes with the Hyflon®AD60 and Teflon®AF1600 layers have only a dioxole-based selective layer.

### Example 2

### Water Permeation with Hyflon®AD60 Selective Layer Only

Samples of the Hyflon®AD membranes of Example 1 were cut into stamps and tested in a permeation test-cell apparatus under pervaporation conditions with ethanol/water mixtures containing different amounts of water. The permeate pressure was maintained at 2.5 ton and the temperature of the feed solution was 75° C. The results are shown in Table 2.

TABLE 2

| Water | Water | Ethanol | |
|---|---|---|---|
| Concentration | Permeance | Permeance | Water/Ethanol |
| in Feed (wt %) | (gpu) | (gpu) | Selectivity |
| 4.7 | 960 | 15 | 64 |
| 17.8 | 1,090 | 17 | 64 |
| 21.2 | 1,060 | 17 | 63 |
| 67.0 | 1,160 | 19 | 61 |
| 86.5 | 1,090 | 16 | 68 |
| 95.7 | 1,370 | 18 | 76 |

As can be seen, the water and ethanol permeances were stable over the tested range, increasing only slightly with increasing water concentrations in the feed solution. The selectivity was also maintained over the range of feed water concentrations, but was only about 60 or 70.

### Example 3

### Water Permeation with Teflon®AF1600 Selective Layer Only

Samples of the Teflon®AF membranes of Example 1 were cut into stamps and tested in a permeation test-cell apparatus under pervaporation conditions with ethanol/water mixtures containing different amounts of water. The test conditions were the same as in Example 2. The results are shown in Table 3.

TABLE 3

| Water | Water | Ethanol | |
|---|---|---|---|
| Concentration | Permeance | Permeance | Water/Ethanol |
| in Feed (wt %) | (gpu) | (gpu) | Selectivity |
| 3.1 | 2,660 | 116 | 23 |
| 4.7 | 2,470 | 108 | 23 |
| 7.2 | 2,970 | 110 | 27 |
| 10.9 | 3,630 | 121 | 30 |
| 17.8 | 2,710 | 100 | 27 |
| 67.0 | 2,940 | 109 | 27 |

As can be seen, this membrane also exhibited good stability under exposure to high concentrations of hot water. The water/ethanol selectivity was considerably lower than for the Hyflon®AD membranes, however.

### Example 4

### Water Permeation with Celfa CMC VP-31 Membrane (Hydrophilic Selective Layer Only.)

Samples of the purchased Celfa CMC VP-31 membranes from Example 1 were cut into stamps and tested in a permeation test-cell apparatus under pervaporation conditions with ethanol/water mixtures containing different amounts of water. The test conditions were the same as in Example 2. The results are shown in Table 4.

TABLE 4

| Water | Water | Ethanol | |
|---|---|---|---|
| Concentration | Permeance | Permeance | Water/Ethanol |
| in Feed (wt %) | (gpu) | (gpu) | Selectivity |
| 3.2 | 3,740 | 8 | 470 |
| 7.4 | 4,310 | 12 | 360 |
| 10.1 | 5,870 | 20 | 290 |
| 15.3 | 7,370 | 29 | 250 |
| 22.5 | 8,650 | 57 | 150 |
| 30.7 | 10,700 | 147 | 73 |

As can be seen, the Celfa membranes exhibited a combination of much higher water permeance and much higher water/ethanol selectivity than the dioxole-based membranes at low water concentrations. The permeances to both water and ethanol increased very substantially as the water concentration in the feed solution increased, indicating swelling of the hydrophilic membrane in the presence of water. The result was a sharp decline in membrane selectivity, from over 300 when the water concentration was below 10 wt % to below 200 when the water concentration was about 20 wt % and below 100 when the water concentration was about 30 wt %.

### Example 5

### Comparison of Hyflon®AD, Teflon®AF and Celfa CMC VP-31 Membranes

Results from test-cell experiments of the type reported in Examples 2-4 were plotted to compare the pervaporation performance of the different membranes. The results are shown in FIG. 12 in the form of a plot of permeate water concentration against feed water concentration. As can be seen, even though this was a simple one-stage experiment, at low feed water concentrations, the Celfa membranes were able to produce a permeate that was mostly water, with only a couple of percent ethanol, an indication of the very high selectivity of the membranes under these conditions. Under the same conditions, both membranes having only dioxole-based selective layers performed well, but could not produce a permeate with a water concentration comparable to the Celfa membranes.

At above about 10 wt % water in the feed, the performance of the Celfa membranes began to drop off sharply, and the Celfa membranes performed less well than the Hyflon®AD membranes after the water concentration in the feed reached about 20 wt % and less well than the Teflon®AF membranes after the water concentration in the feed reached about 25 wt %.

The Hyflon®AD membranes could produce a permeate containing less than 18 wt % ethanol across the entire range of water concentrations.

The experiments were repeated with butanol/water mixtures and similar results were obtained.

### Example 6

### Celfa CMC VP 31/Hyflon®AD Membranes in Accordance with the Invention

Celfa CMC VP 31 membranes as purchased were dip-coated in Hyflon®AD60 solutions of different polymer concentrations and dried in an oven at 60° C. for 10 minutes, to yield membranes of the type shown in FIG. 5, having both a hydrophilic selective layer and a dioxole-based selective layer.

The coating solution concentration was varied from 0.25 wt % to 1 wt %. The membranes had dioxole-based selective layers of different thicknesses, depending on the concentration of Hyflon®AD in the coating solution.

Samples of the membranes were cut into stamps and tested in a permeation test cell apparatus, following the procedure described above for Example 2. The results are shown in Tables 5-7.

TABLE 5
Membrane made with coating solution concentration
of 0.25 wt % Hyflon ®AD60

| Water | Water | Ethanol | |
|---|---|---|---|
| Concentration | Permeance | Permeance | Water/Ethanol |
| in Feed (wt %) | (gpu) | (gpu) | Selectivity |
| 9.78 | 6,420 | 14 | 450 |
| 21.9 | 9,910 | 44 | 220 |
| 50.0 | 21,140 | 1,270 | 17 |
| 86.0 | 27,000 | 5,460 | 5 |

TABLE 6
Membrane made with coating solution concentration
of 0.5 wt % Hyflon ®AD60

| Water | Water | Ethanol | |
|---|---|---|---|
| Concentration | Permeance | Permeance | Water/Ethanol |
| in Feed (wt %) | (gpu) | (gpu) | Selectivity |
| 9.78 | 3,720 | 8 | 490 |
| 21.9 | 5,330 | 13 | 407 |
| 50.0 | 7,110 | 65 | 110 |
| 86.0 | 8,640 | 400 | 22 |

TABLE 7
Membrane made with coating solution concentration
of 1.0 wt % Hyflon ®AD60

| Water | Water | Ethanol | |
|---|---|---|---|
| Concentration | Permeance | Permeance | Water/Ethanol |
| in Feed (wt %) | (gpu) | (gpu) | Selectivity |
| 9.78 | 2,890 | 6 | 490 |
| 21.9 | 2,430 | 6 | 380 |
| 50.0 | 3,700 | 23 | 160 |
| 86.0 | 3,720 | 91 | 4 |

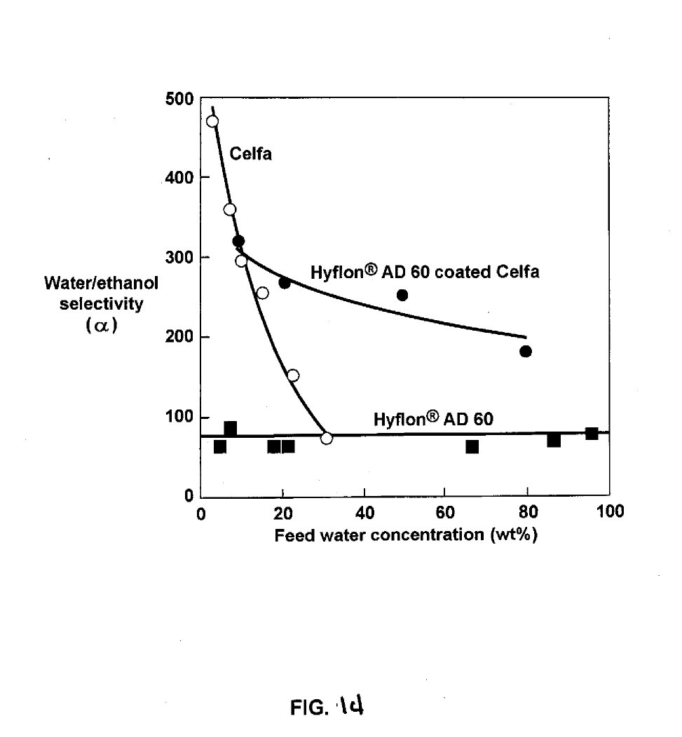

FIG. 13 is a plot comparing the data from Tables 5-7 with results obtained from Celfa membranes without a Hyflon®AD layer. As can be seen, the membranes with the thinnest Hyflon®AD layer showed essentially the same water permeance as the uncoated Celfa membranes, indicating that the layer was too thin to influence the water permeation properties. The membrane with the thickest dioxole-based selective layer exhibited the most stable performance over the range of water concentrations in terms of water permeance. In other words, the thickest layer best protected the Celfa membrane from swelling, while still providing high permeability to water.

### Example 7

### Membrane Selectivity Performance Comparison

Samples of three membranes types were prepared:

(i) Celfa CMC VP 31 as purchased;

(ii) 0.5 wt % Hyflon®AD60 selective layer, prepared as in Example 1;

(iii) 0.5 wt % Hyflon®AD60 on purchased Celfa CMC VP 31, prepared as in Example 6.

Only membrane type (iii) was in accordance with the invention.

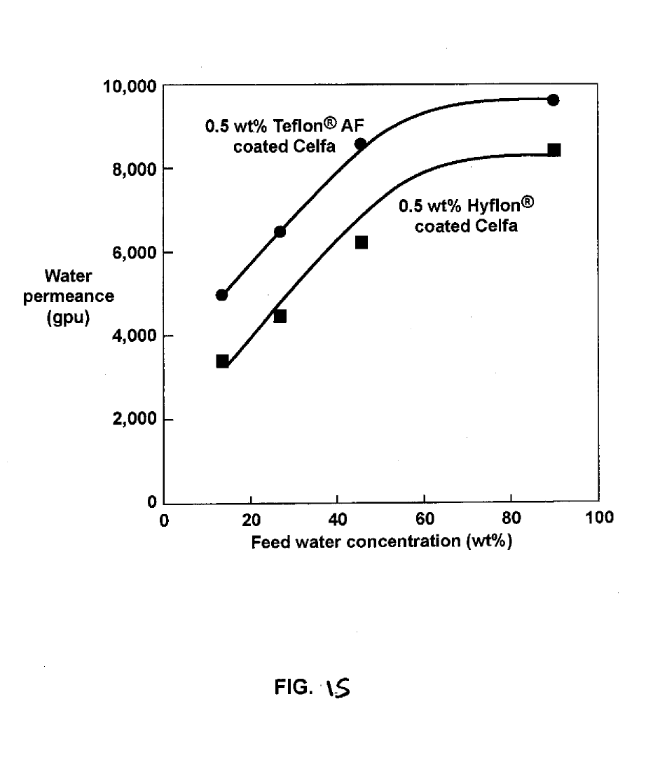

Samples of the membranes were cut into stamps and tested in a permeation test cell apparatus, following the procedure described above for Example 2. The results are shown in Table 8 and FIG. 14.

TABLE 8

| | Water | Water | Ethanol | Water/ |
|---|---|---|---|---|
| Membrane | Concentration | Permeance | Permeance | Ethanol |
| Type | in Feed (wt %) | (gpu) | (gpu) | Selectivity |
| (i) Hydrophilic | 7 | 4310 | 12 | 370 |
| selective | 22 | 8,640 | 57 | 150 |
| layer only | 31 | 10,670 | 147 | 70 |
| (ii) Dioxole- | 7 | 770 | 9 | 90 |
| based selective | 28 | 1,110 | 18 | 60 |
| layer only | 67 | 1,160 | 19 | 60 |
| (iii) Hydrophilic | 10 | 3,600 | 11 | 320 |
| and dioxole-based | 21 | 5,650 | 21 | 270 |
| selective layers | 50 | 5,890 | 22 | 260 |

As can be seen, the membranes having only a hydrophilic selective layer outperform the other membranes with respect to water/ethanol selectivity at low water concentrations. The membranes having only a dioxole-based selective layer exhibit much more stable water/ethanol selectivity, and match the selectivity of the hydrophilic membranes when the water content of the feed reaches about 30 wt %.

At all water concentrations above about 10 wt %, the membranes having both a hydrophilic selective layer and a dioxole-based selective layer exhibit higher selectivity than either the hydrophilic Celfa membrane or the dioxole-based Hyflon®AD membrane. Furthermore, this selectivity remains reasonably stable and high, at 200 or above, even when the feed solution contains 80 wt % water. Neither of the other membranes come close to this performance, as both have a selectivity less than 100 at high water concentrations.

### Example 8

### Comparison of Membranes Using Hyflon®AD and Teflon®AF as Dioxole-Based Selective Layer

Two sets of membranes with a hydrophilic selective layer and a dioxole-based selective layer were made by coating purchased Celfa CMC VP 31 membranes using either a single coating of a solution containing 0.5 wt % Teflon®AF or 0.5 wt % Hyflon®AD60.

Samples of the membranes were cut into stamps and tested in a permeation test cell apparatus, following the procedure described above for Example 2. The results are shown as a plot of water permeance of the membranes against feed water concentration in FIG. 15. As can be seen, the membranes with the Teflon®AF coating show higher water permeance than those with the Hyflon®AD coating over the range of water concentrations in the feed. For each membrane, the water permeance roughly doubles from 10 wt % to 90 wt % feed water concentration.

### Example 9

### Process Calculations for Stripping/Membrane Hybrid Process

A computer calculation was performed to simulate the performance of a process of the type shown in FIG. 4 in separating water from ethanol. The calculation was carried out a modeling program, ChemCad V (ChemStations, Inc., Houston, Tex.), modified with MTR proprietary code. The feed stream to the process was assumed to be a solution of 11.5 wt % ethanol in water; the goal was to produce a dehydrated ethanol stream with an ethanol concentration of 99.7 wt % ethanol, such as would be suitable as fuel-grade ethanol.

The process uses a stripping step followed by a membrane separation step, as in FIG. 8. The stripping step was assumed to be performed as in a beer still, with no condensation/rectification for the overhead vapor from the column. In this case, the membrane separation step was assumed to be performed in two sub-steps. Each sub-step was assumed to use Celfa CMC VP 31 membranes with an additional selective layer of Hyflon®AD60, prepared as in Example 7. In the alternative, if the feed to the second sub-step contains a relatively low concentration of water, it is possible to use a membrane with only the hydrophilic selective layer for the second sub-step.

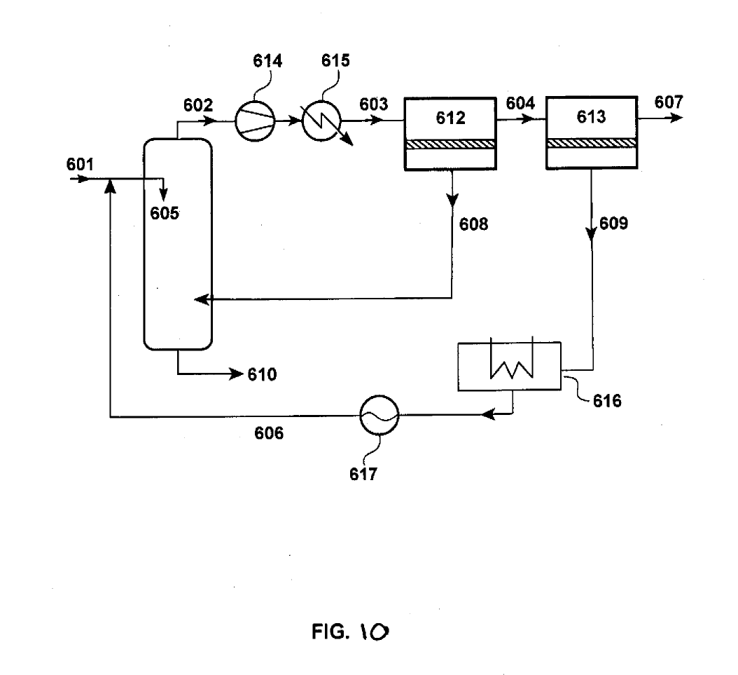

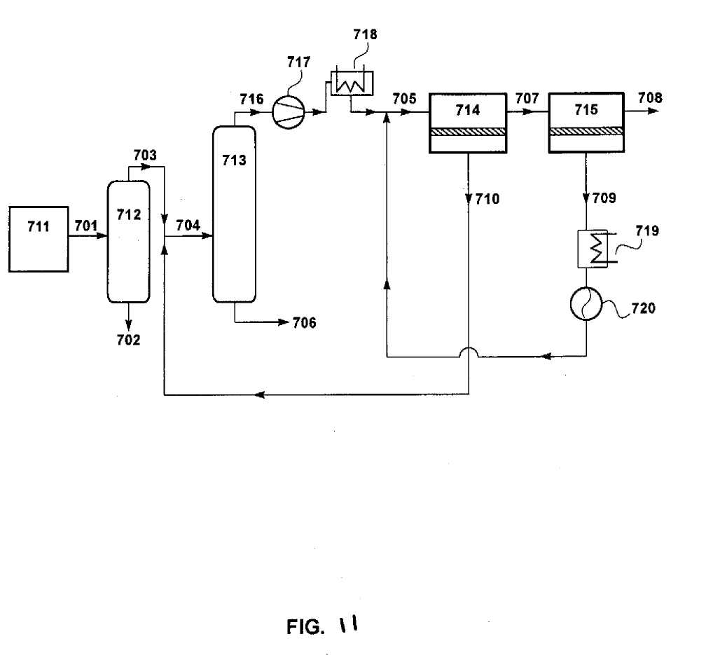

The process flow diagram is shown in FIG. 10. Referring to this figure, liquid feed stream, 601, enters stripping column or beer still, 605, which operates at the suction pressure of compressor, 614, that is, half an atmosphere pressure.

Ethanol-enriched vapor stream, 602, is withdrawn from the top of the column, and water stream, 610, is withdrawn from the bottom, after passing through the reboiler (not shown).

The overhead stream from the column passes through compressor, 614, and is cooled, 615, before entering the first membrane separation step, 612, as membrane feed stream, 603. This step uses about 1,600 m² of membrane area to reduce the water content of the process stream to about 10 wt %. Water preferentially permeates the membranes and emerges from the permeate side as first permeate vapor stream, 608. This stream is returned to the stripping column. The first dehydrated residue vapor is withdrawn as residue stream, 604, and passes as feed to the second membrane separation step, 613, which uses about 5,000 m² of membrane area.

The residue stream, 607, from this step is the dehydrated ethanol product of the process, containing 99.7 wt % ethanol. The second permeate stream, 609, is condensed, 616, and pumped by liquid pump, 617, to return to the beer still as stream, 606.

The results of the calculation are shown in Table 9. As can be seen, the process produces a high-quality ethanol product and a water stream with very little ethanol.

TABLE 9

| | Process | Column | Membrane | Water | Dehydrated | First | Ethanol | Recycle | Condensed |
|---|---|---|---|---|---|---|---|---|---|
| | feed | overhead | feed | stream | residue | permeate | product | stream | recycle |
| Stream | 601 | 602 | 603 | 610 | 604 | 608 | 607 | 609 | stream 606 |
| Flux | 165,000 | 32,650 | 32,650 | 146,111 | 22,207 | 11,443 | 18,888 | 3,318 | 3,318 |
| (kg/h) | | | | | | | | | |
| Temp. | 37 | 70 | 120 | 81 | 116 | 118 | 114 | 30 | 32 |
| (° C.) | | | | | | | | | |
| Pressure | 1.0 | 0.5 | 3.0 | 0.5 | 3.0 | 0.5 | 3.0 | 0.1 | 1.0 |
| (bar) | | | | | | | | | |
| Water | 88.5 | 36.3 | 36.3 | 99.9 | 9.9 | 92.5 | 0.3 | 64.7 | 64.7 |
| (wt %) | | | | | | | | | |
| Ethanol | 11.5 | 63.7 | 63.7 | 90.1 | 90.1 | 7.5 | 99.7 | 35.3 | 35.3 |
| (wt %) | | | | | | | | | |

### Example 10

### Process Calculations for Bioethanol Production Process

A computer calculation was performed to simulate the performance of a process of the type shown in FIG. 5 to produce ethanol from biomass. The calculation was again carried out using ChemCad V. The fermentation step was not modeled, but was assumed to produce a solution containing 11.5 wt % ethanol in water, as might be produced from conventional fermentation of corn, for example.

The membrane separation step was assumed to be performed in two sub-steps. Each sub-step was assumed to use Celfa CMC VP 31 membranes with an additional selective layer of Hyflon®AD60, prepared as in Example 7. In the alternative, the second sub-step, which is exposed to only a low water concentration in its feed stream, could be carried out using a membrane having only a hydrophilic selective layer.

The process flow diagram is shown in FIG. 11. Referring to this figure, fermentation step, 711, yields stream, 701, containing 11.5 wt % ethanol. This stream enters beer still, 712, and is separated into water stream, 702, and overhead vapor stream, 703. The overhead stream from the stripper is mixed with return stream, 710, and enters distillation or rectification column, 713, as stream, 704. Both the stripper and the rectification column operate at half an atmosphere pressure, created by the suction of compressor, 717.

The distillation step produces an overhead stream, 716, containing about 93 wt % ethanol. Because the membrane separation steps are relied on for the final purification of the ethanol product, the distillation column overhead need not be driven all the way to the azeotrope. The bottoms stream, 706, from this column, like the bottoms stream from the stripper, contains very little ethanol.

The overhead from the distillation column is compressed, 717, condensed, 718, and mixed with return stream, 709, to be sent as a feed stream, 705, after heating to provide transmembrane driving force (not shown), to the first membrane separation step, 714. This step uses about 1,200 m² of membrane area.

Water preferentially permeates the membranes and emerges from the permeate side as first permeate vapor stream, 710. This stream is recirculated to be mixed with stream 703 as feed to the rectification column. The first dehydrated residue vapor is withdrawn as residue stream, 707, and passes as feed to the second membrane separation step, 715, which uses about 4,400 m² of membrane area.

The residue stream, 708, from this step is the dehydrated ethanol product, containing 99.7 wt % ethanol. The permeate stream, 709, is condensed, 719, and pumped by liquid pump, 720, to return to the front of the membrane separation unit.

The results of the calculation are shown in Table 10. Once again, the process produces a high-quality ethanol product and a water stream with very little ethanol.

TABLE 10

| | Process | Overhead | Water | Overhead | Membrane | Water | Dehydrated | Ethanol | Recycle to | Recycle to |
|---|---|---|---|---|---|---|---|---|---|---|
| | feed | (still) | stream | (column) | feed | stream | Residue | product | membrane | column |
| Stream | 701 | 703 | 702 | 716 | 705 | 706 | 707 | 708 | 709 | 710 |
| Flux | 165,000 | 35,273 | 129,726 | 20,750 | 22,412 | 16,387 | 20,548 | 18,885 | 1,663 | 1,864 |
| (kg/h) | | | | | | | | | | |
| Temp. | 37 | 73 | 81 | 61 | 115 | 81 | 115 | 42 | 110 | 110 |
| (° C.) | | | | | | | | | | |
| Pressure | 1.0 | 0.5 | 0.5 | 0.5 | 4.0 | 0.5 | 4.0 | 4.0 | 0.1 | 0.2 |
| (bar) | | | | | | | | | | |
| Water | 88.5 | 46.6 | 99.9 | 7.0 | 9.0 | 99.9 | 3.0 | 0.3 | 33.7 | 74.9 |
| (wt %) | | | | | | | | | | |
| Ethanol | 11.5 | 53.4 | 0.1 | 93.0 | 91.0 | 0.1 | 97.0 | 99.7 | 66.3 | 25.1 |
| (wt %) | | | | | | | | | | |

We claim:

1. A process for separating water from organic compounds comprising:
(a) providing a composite membrane having a feed side and a permeate side, the composite membrane comprising:
(i) a microporous support layer;
(ii) a first dense selective layer of a hydrophilic polymer; and
(iii) a second dense selective layer of a dioxole-based polymer having the structure 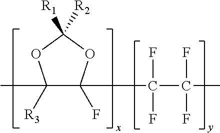
wherein R₁ and R₂ are fluorine or CF₃, R₃ is fluorine or —O—CF₃, and x and y represent the relative proportions of the dioxole and the tetrafluoroethylene blocks, such that x+y=1;
the first dense selective layer being positioned between the microporous support layer and the second dense selective layer;
(b) passing a feed solution comprising water and an organic compound across the feed side;
(c) withdrawing from the feed side a dehydrated solution having a lower water content than that of the feed solution;
(d) withdrawing from the permeate side a permeate vapor having a higher water content than that of the feed solution.

2. The process of claim 1, wherein the hydrophilic polymer is polyvinyl alcohol.

3. The process of claim 1, wherein the hydrophilic polymer is a cellulose derivative.

4. The process of claim 1, wherein the dioxole-based polymer has the structure 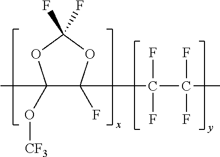
where x and y represent the relative proportions of the dioxole and the tetrafluoroethylene blocks, such that x+y=1.

5. The process of claim 1, wherein the dioxole-based polymer has the structure 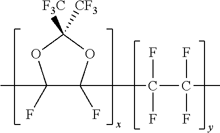
where x and y represent the relative proportions of the dioxole and the tetrafluoroethylene blocks, such that x+y=1.

6. The process of claim 1, wherein the feed solution has a water content of at least 10 wt %.

7. The process of claim 1, wherein the feed solution has a water content of at least 20 wt %.

8. The process of claim 1, wherein the feed solution has a water content of at least 50 wt %.

9. The process of claim 1, wherein the feed solution is at a temperature of at least about 60° C.

10. The process of claim 1, wherein the organic compound is chosen from the group consisting of methanol, ethanol, isopropanol, butanol, acetone, acetic acid, and formaldehyde.

11. The process of claim 1, wherein the organic compound is ethanol.

12. The process of claim 1, in which the composite membrane exhibits a higher water/organic compound selectivity than is exhibited by either (a) a first membrane having only a hydrophilic polymer selective layer of the same hydrophilic polymer as the first dense selective layer, or (b) a second membrane having only a dioxole-based polymer selective layer of the same dioxole-based polymer as the second dense selective layer.

13. The process of claim 1, further comprising passing the dehydrated solution across a second composite membrane to create a dehydrated product solution that has a lower water content than that of the dehydrated solution.

14. A process for separating water from organic compounds comprising:
(a) providing a composite membrane having a feed side and a permeate side, the membrane comprising:
(i) a microporous support layer;
(ii) a first dense selective layer of a hydrophilic polymer; and
(iii) a second dense selective layer of a dioxole-based polymer having the structure 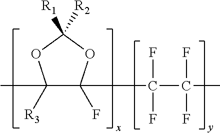
wherein R₁ and R₂ are fluorine or CF₃, R₃ is fluorine or —O—CF₃, and x and y represent the relative proportions of the dioxole and the tetrafluoroethylene blocks, such that x+y=1;
the first dense selective layer being positioned between the microporous support layer and the second dense selective layer;
(b) passing a feed vapor comprising water and an organic compound across the feed side;
(c) withdrawing from the feed side a dehydrated vapor having a water content lower than that of the feed solution;
(d) withdrawing from the permeate side a permeate vapor having a higher water content than the feed solution.

15. The process of claim 14, wherein the hydrophilic polymer is polyvinyl alcohol.

16. The process of claim 14, wherein the hydrophilic polymer is a cellulose derivative.

17. The process of claim 14, wherein the dioxole-based polymer has the structure 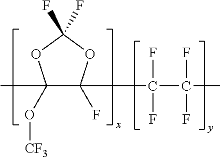
where x and y represent the relative proportions of the dioxole and the tetrafluoroethylene blocks, such that x+y=1.

18. The process of claim 14, wherein the dioxole-based polymer has the structure 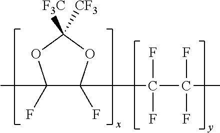
where x and y represent the relative proportions of the dioxole and the tetrafluoroethylene blocks, such that x+y=1.

19. The process of claim 14, wherein the feed vapor has a water content of at least 10 wt %.

20. The process of claim 14, wherein the feed vapor has a water content of at least 20 wt %.

21. The process of claim 14, wherein the feed vapor has a water content of at least 50 wt %.

22. The process of claim 14, wherein the organic compound is ethanol.

23. The process of claim 14, in which the composite membrane exhibits a higher water/organic compound selectivity than is exhibited by either (a) a first membrane having only a hydrophilic polymer selective layer of the same hydrophilic polymer as the first dense selective layer, or (b) a second membrane having only a dioxole-based polymer selective layer of the same dioxole-based polymer as the second dense selective layer.

24. The process of claim 14, further comprising passing the dehydrated vapor across a second composite membrane to create a dehydrated product vapor that has a lower water content than that of the dehydrated vapor.

25. A composite membrane having a feed side and a permeate side, the membrane comprising:
(i) a microporous support layer;
(ii) a first dense selective layer of a hydrophilic polymer; and
(iii) a second dense selective layer of a dioxole-based polymer having the structure 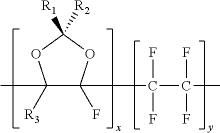
wherein R₁ and R₂ are fluorine or CF₃, R₃ is fluorine or —O—CF₃, and x and y represent the relative proportions of the dioxole and the tetrafluoroethylene blocks, such that x+y=1;
the first dense selective layer being positioned between the microporous support layer and the second dense selective layer;
wherein, when challenged with a feed solution containing 20 wt % water at a set of operating conditions that include a feed solution temperature of 75° C., the composite membrane has a higher water/organic compound selectivity than that of either (a) a first membrane having only a hydrophilic polymer selective layer of the same hydrophilic polymer as the first dense selective layer, or (b) a second membrane having only a dioxole-based polymer selective layer of the same dioxole-based polymer as the second dense selective layer, all as measured at the set of operating conditions.

26. The composite membrane of claim 25, wherein the hydrophilic polymer is chosen from the group consisting of cellulose derivatives and polyvinyl alcohol.

27. The composite membrane of claim 25, wherein the dioxole-based polymer has the structure 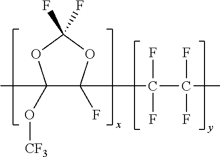
where x and y represent the relative proportions of the dioxole and the tetrafluoroethylene blocks, such that x+y=1.

28. The composite membrane of claim 25, wherein the dioxole-based polymer has the structure 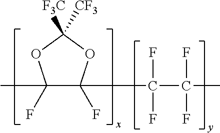
where x and y represent the relative proportions of the dioxole and the tetrafluoroethylene blocks, such that x+y=1.

29. A stripping/membrane separation process for separating water from organic compounds comprising:
(a) subjecting a feed solution comprising water and an organic compound to a stripping step, thereby producing an organic-compound-enriched overhead vapor stream and an organic-compound-depleted bottoms stream;
(b) subjecting the overhead vapor stream to a membrane separation step comprising:
(I) providing a composite membrane having a feed side and a permeate side, the membrane comprising:
(i) a microporous support layer;
(ii) a first dense selective layer of a hydrophilic polymer; and
(iii) a second dense selective layer of a dioxole-based polymer having the structure 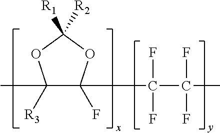
wherein R₁ and R₂ are fluorine or CF₃, R₃ is fluorine or —O—CF₃, and x and y represent the relative proportions of the dioxole and the tetrafluoroethylene blocks, such that x+y=1;
the first dense selective layer being positioned between the microporous support layer and the second dense selective layer;
(II) passing the overhead feed vapor across the feed side;
(III) withdrawing from the feed side a dehydrated vapor having a water content lower than that of the overhead feed vapor;
(IV) withdrawing from the permeate side a permeate vapor having a higher water content than that of the overhead feed vapor.

30. The stripping/membrane separation process of claim 29, wherein the organic compound comprises ethanol.

31. The stripping/membrane separation process of claim 29, wherein the dioxole-based polymer has the structure 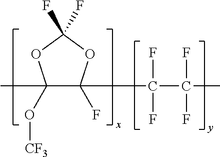
where x and y represent the relative proportions of the dioxole and the tetrafluoroethylene blocks, such that x+y=1.

32. The stripping/membrane separation process of claim 29, wherein the dioxole-based polymer has the structure 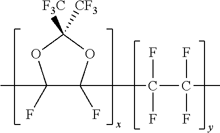
where x and y represent the relative proportions of the dioxole and the tetrafluoroethylene blocks, such that x+y=1.

33. The stripping/membrane separation process of claim 29, further comprising passing the dehydrated vapor across a second composite membrane to create a dehydrated product vapor that has a lower water content than that of the dehydrated vapor.

34. An ethanol production process, comprising the following steps:
(a) fermenting a biomass to produce ethanol;
(b) subjecting an ethanol-containing stream from step (a) to a first separation step to increase the ethanol concentration by at least three-fold to produce an ethanol-enriched stream;
(c) subjecting the ethanol-enriched stream to a second separation step to further enrich the ethanol concentration to produce an ethanol-rich stream and an ethanol-lean stream;
(d) subjecting the ethanol-rich stream to a dehydration step using a composite membrane having a feed side and a permeate side, the membrane comprising:
(i) a microporous support layer;
(ii) a first dense selective layer of a hydrophilic polymer; and
(iii) a second dense selective layer of a dioxole-based polymer having the structure 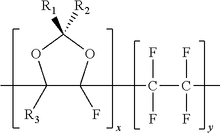
wherein R₁ and R₂ are fluorine or CF₃, R₃ is fluorine or —O—CF₃, and x and y represent the relative proportions of the dioxole and the tetrafluoroethylene blocks, such that x+y=1;
the first dense selective layer being positioned between the microporous support layer and the second dense selective layer, thereby producing a dehydrated ethanol product.

35. The ethanol production process of claim 34, wherein the ethanol-rich stream is sent to the dehydration step as a vapor.

36. The ethanol production process of claim 34, wherein the ethanol-containing stream has an ethanol concentration less than 15 wt %, the ethanol-enriched stream has an ethanol concentration of at least 50 wt % and the dehydrated ethanol product has an ethanol concentration of at least 99 wt %.

37. The ethanol production process of claim 34, wherein the dehydration step is performed in two sub-steps.

38. The ethanol production process of claim 34, wherein the first separation step comprises a steam-stripping step.

39. The ethanol production process of claim 34, wherein the second separation step comprises a distillation step.

40. A process for separating water from organic compounds comprising:
(a) providing a composite membrane having a feed side and a permeate side, the composite membrane comprising:
(i) a microporous support layer;
(ii) a first dense selective layer of a hydrophilic polymer; and
(iii) a second dense selective layer of a polymer having the structure 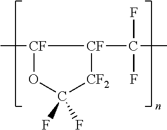
where n is a positive integer;
the first dense selective layer being positioned between the microporous support layer and the second dense selective layer;
(b) passing a feed mixture comprising water and an organic compound across the feed side;
(c) withdrawing from the feed side a dehydrated mixture having a lower water content than that of the feed mixture;
(d) withdrawing from the permeate side a permeate vapor having a higher water content than that of the feed mixture.

41. The process of claim 40, wherein the organic compound is ethanol.
